SEQUENCE LISTING (24 sequences)

SEQ ID NO: 1 - DNA, 60 bp, Hepatitis C virus
gaattcgcggccgccatgagcaccaaccccaagccccagcgcaagaccaagcggaacacc

SEQ ID NO: 2 - DNA, 59 bp, Hepatitis C virus
gaattcggatcctcatgcgctagcggggatggtgaggcagctcagcagcgccagcagga

SEQ ID NO: 3 - DNA, 55 bp, Hepatitis C virus
gaattcgcggccgccatggcccccatcaccgcctacagccagcagacccggggac

SEQ ID NO: 4 - DNA, 55 bp, Hepatitis C virus
gaattcggatcctcaggtgaccacctccaggtcagcggacatgcacgccatgatg

SEQ ID NO: 5 - DNA, 46 bp, Hepatitis C virus
gaattcgcggccgccatgttttgggccaagcatatgtggaacttca

SEQ ID NO: 6 - DNA, 46 bp, Hepatitis C virus
gaattcggatcctcagcaaggggtggagcagtcctcgttgatccac

SEQ ID NO: 7 - DNA, 49 bp, Hepatitis C virus
gaattcgcggccgccatgtccatgtcctacacctggaccggcgccctga

SEQ ID NO: 8 - DNA, 49 bp, Hepatitis C virus
gaattcggatcctcagcggttgggcagcaggtagatgccgactccgacg

SEQ ID NO: 9 - protein, 191 aa, Hepatitis C virus
MSTNPKPQRKTKRNTNRRPQDVKFPGGGQIVGGVYLLPRRGPRLGVRATRKTSERSQPRGRRQPIPKARRPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASA

SEQ ID NO: 10 - protein, 632 aa, Hepatitis C virus
MAPITAYSQQTRGLLGCIITSLTGRDKNQVEGEVQVVSTATQSFLATCINGVCWTVYHGAGSKTLAGPKGPITQMYTNVDQDLVGWQAPPGARSMTPCTCGSSDLYLVTRHADVIPVRRRGDSRGSLLSPRPVSYLKGSVGGPLLCPSGHVVGIFRAAVCTRGVAKAVDFIPVESMETTMRSPVFTDNSSPPAVPQTFQVAHLHAPTGSGKSTKVPAAYAAQGYKVLVLNPSVAATLGFGAYMSKAHGIDPNIRTGVRTITTGAPITYSTYGKFLADGGCSGGAYDIIICQECHSTDSTTILGIGTVLDQAETAGARLVVLATATPPGSVTVPHPNIEEVALSNNGEIPFYGKAIPIEAIKGGRHLIFCHSKKKCDELAAKLSGLGLNAVAYYRGLDVSVIPTSGDVVVVATDALMTGFTGDFDSVIDCNTCVTQTVDFSLDPTFTIETTTVPQDAVSRSQRRGRTGRGRSGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTLTHPITKYIMACMSADLEVVT

SEQ ID NO: 11 - protein, 214 aa, Hepatitis C virus
MFWAKHMWNFISGIQYLAGLSTLPGNPAIASLMAFTASITSPLTTQNTLLFNILGGWVAAQLAPPSAASAFVGAGIAGAAVGSIGLGKVLVDILAGYGAGVAGALVAFKVMSGEVPSTEDLVNLLPAILSPGALVVGVVCAAILRRHVGPGEGAVQWMNRLIAFASRGNHVSPTHYVPESDAAARVTQILSSLTITQLLKRLHQWINEDCSTPC

SEQ ID NO: 12 - protein, 592 aa, Hepatitis C virus
MSMSYTWTGALITPCAAEESKLPINPLSNSLLRHHNMVYATTSRSASLRQKKVTFDRLQVLDDHYRDVLKEMKAKASTVKAKLLSIEEACKLTPPHSAKSKFGYGAKDVRNLSSRAVNHIRSVWEDLLEDTETPIDTTIMAKSEVFCVQPEKGGRKPARLIVFPDLGVRVCEKMALYDVVSTLPQAVMGSSYGFQYSPKQRVEFLVNTWKSKKCPMGFSYGTRCFGSTVTESDIRVEESIYQCCDLAPEARQAIRSLTERLYIGGPLTNSKGQNCGYRRCRASGVLTTSCGNTLTCYLKATAACRAAKLQDCTMLVNGDDLVVICESAGTQEDAAALRAFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRAKLLSQGGRAATCGRYLFNWAVRTKLKLTPIPAASQLDLSGWFVAGYSGGDIYHSLSRARPRWFPLCLLLLSVGVGIYLLPNR

SEQ ID NO: 13 - protein, 15 aa, Hepatitis C virus
PRFGKAIPIEAIKGG

SEQ ID NO: 14 - protein, 15 aa, Hepatitis C virus
YRLGAVQNEVILTHP

SEQ ID NO: 15 - protein, 15 aa, Hepatitis C virus
SMSYTWTGALITPCA

SEQ ID NO: 16 - protein, 15 aa, Hepatitis C virus
AAALRAFTEAMTRYS

SEQ ID NO: 17 - protein, 15 aa, Hepatitis C virus
IQYLAGLSTLPGNPA

SEQ ID NO: 18 - protein, 15 aa, Hepatitis C virus
FWAKHMWNFISGIWY

SEQ ID NO: 19 - DNA, 9595 bp, Hepatitis C virus
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcaagttcccgggcggtggtcagatcgttggtggagtttacctgttgccgcgcaggggccccaggttgggtgtgcgcgcgactaggaaggcttccgagcggtcgcaacctcgtggaaggcgacaacctatcccaaaggctcgccgacccgagggcagggcctgggctcagcccgggtacccttggcccctctatggcaatgagggcctggggtgggcaggatggctcctgtcaccccgcggctcccggcctagttggggccccacggacccccggcgtaggtcgcgtaacttgggtaaggtcatcgatacccttacatgcggcttcgccgatctcatggggtacattccgctcgtcggcgcccccctagggggcgctgccagggccttggcacacggtgtccgggttctggaggacggcgtgaactatgcaacagggaacttgcccggttgctctttctctatcttcctcttggctctgctgtcctgtttgaccatcccagcttccgcttatgaagtgcgcaacgtgtccgggatataccatgtcacgaacgactgctccaactcaagcattgtgtatgaggcagcggacgtgatcatgcatactcccgggtgcgtgccctgtgttcaggagggtaacagctcccgttgctgggtagcgctcactcccacgctcgcggccaggaatgccagcgtccccactacgacaatacgacgccacgtcgacttgctcgttgggacggctgctttctgctccgctatgtacgtgggggatctctgcggatctattttcctcgtctcccagctgttcaccttctcgcctcgccggcatgagacagtgcaggactgcaactgctcaatctatcccggccatgtatcaggtcaccgcatggcttgggatatgatgatgaactggtcacctacaacagccctagtggtgtcgcagttgctccggatcccacaagctgtcgtggacatggtggcgggggcccactggggagtcctggcgggccttgcctactattccatggtagggaactgggctaaggttctgattgtggcgctactctttgccggcgttgacggggagacccacacgacggggagggtggccggccacaccacctccgggttcacgtcccttttctcatctggggcgtctcagaaaatccagcttgtgaataccaacggcagctggcacatcaacaggactgccctaaattgcaatgactccctccaaactgggttctttgccgcgctgttttacgcacacaagttcaactcgtccgggtgcccggagcgcatggccagctgccgccccattgactggttcgcccaggggtggggccccatcacctatactaagcctaacagctcggatcagaggccttattgctggcattacgcgcctcgaccgtgtggtgtcgtacccgcgtcgcaggtgtgtggtccagtgtattgtttcaccccaagccctgttgtggtggggaccaccgatcgttccggtgtccctacgtatagctggggggagaatgagacagacgtgatgctcctcaacaacacgcgtccgccacaaggcaactggttcggctgtacatggatgaatagtactgggttcactaagacgtgcggaggtcccccgtgtaacatcgggggggtcggtaaccgcaccttgatctgccccacggactgcttccggaagcaccccgaggctacttacacaaaatgtggctcggggccctggttgacacctaggtgcctagtagactacccatacaggctttggcactacccctgcactctcaatttttccatctttaaggttaggatgtatgtggggggcgtggagcacaggctcaatgccgcatgcaattggactcgaggagagcgctgtaacttggaggacagggataggtcagaactcagcccgctgctgctgtctacaacagagtggcagatactgccctgtgctttcaccaccctaccggctttatccactggtttgatccatctccatcagaacatcgtggacgtgcaatacctgtacggtgtagggtcagcgtttgtctcctttgcaatcaaatgggagtacatcctgttgcttttccttctcctggcagacgcgcgcgtgtgtgcctgcttgtggatgatgctgctgatagcccaggctgaggccgccttagagaacttggtggtcctcaatgcggcgtccgtggccggagcgcatggtattctctcctttcttgtgttcttctgcgccgcctggtacattaagggcaggctggctcctggggcggcgtatgctttttatggcgtatggccgctgctcctgctcctactggcgttaccaccacgagcttacgccttggaccgggagatggctgcatcgtgcgggggtgcggttcttgtaggtctggtattcttgaccttgtcaccatactacaaagtgtttctcactaggctcatatggtggttacaatactttatcaccagagccgaggcgcacatgcaagtgtgggtcccccccctcaacgttcggggaggccgcgatgccatcatcctcctcacgtgtgcggttcatccagagttaatttttgacatcaccaaactcctgctcgccatactcggcccgctcatggtgctccaggctggcataacgagagtgccgtacttcgtgcgcgctcaagggctcattcgtgcatgcatgttagtgcgaaaagtcgccgggggtcattatgtccaaatggtcttcatgaagctgggcgcgctgacaggtacgtacgtttataaccatcttaccccactgcgggactgggcccacgcgggcctacgagaccttgcggtggcggtagagcccgtcgtcttctccgccatggagaccaaggtcatcacctggggagcagacaccgctgcgtgtggggacatcatcttgggtctacccgtctccgcccgaagggggaaggagatatttttgggaccggctgatagtctcgaagggcaagggtggcgactccttgcgcccatcacggcctactcccaacaaacgcggggcgtacttggttgcatcatcactagcctcacaggccgggacaagaaccaggtcgaaggggaggttcaagtggtttctaccgcaacacaatctttcctggcgacctgcatcaacggcgtgtgctggactgtctaccatggcgctggctcgaagaccctagccggtccaaaaggtccaatcacccaaatgtacaccaatgtagacctggacctcgtcggctggcaggcgccccccggggcgcgctccatgacaccatgcagctgtggcagctcggacctttacttggtcacgagacatgctgatgtcattccggtgcgccggcgaggcgacagcaggggaagtctactctcccccaggcccgtctcctacctgaaaggctcctcgggtggtccattgctttgcccttcggggcacgtcgtgggcgtcttccgggctgctgtgtgcacccggggggtcgcgaaggcggtggacttcatacccgttgagtctatggaaactaccatgcggtctccggtcttcacagacaactcaacccccccggctgtaccgcagacattccaagtggcacatctgcacgctcctactggcagcggcaagagcaccaaagtgccggctgcgtatgcagcccaagggtacaaggtgctcgtcctgaacccgtccgttgccgccaccttagggtttggggcgtatatgtccaaggcacacggtatcgaccctaacatcagaactggggtaaggaccattaccacgggcggctccattacgtactccacctatggcaagttccttgccgacggtggctgttctgggggcgcctatgacatcataatatgtgatgagtgccactcaactgactcgactaccatcttgggcatcggcacagtcctggaccaagcggagacggctggagcgcggctcgtcgtgctcgccaccgctacacctccgggatcggttaccgtgccacaccccaatatcgaggaaataggcctgtccaacaatggagagatccccttctatggcaaagccatccccattgaggccatcaagggggggaggcatctcattttctgccattccaagaagaaatgtgacgagctcgccgcaaagctgacaggcctcggactgaacgctgtagcatattaccggggccttgatgtgtccgtcataccgcctatcggagacgtcgttgtcgtggcaacagacgctctaatgacgggtttcaccggcgattttgactcagtgatcgactgcaatacatgtgtcacccagacagtcgacttcagcttggatcccaccttcaccattgagacgacgaccgtgccccaagacgcggtgtcgcgctcgcaacggcgaggtagaactggcaggggtaggagtggcatctacaggtttgtgactccaggagaacggccctcgggcatgttcgattcttcggtcctgtgtgagtgctatgacgcgggctgtgcttggtatgagctcacgcccgctgagacctcggttaggttgcgggcttacctaaatacaccagggttgcccgtctgccaggaccatctggagttctgggagagcgtcttcacaggcctcacccacatagatgcccacttcctgtcccagactaaacaggcaggagacaactttccttacctggtggcatatcaagctacagtgtgcgccagggctcaagctccacctccatcgtgggaccaaatgtggaagtgtctcatacggctgaaacctacactgcacgggccaacacccctgctgtataggctaggagccgtccaaaatgaggtcatcctcacacaccccataactaaatacatcatggcatgcatgtcggctgacctggaggtcgtcactagcacctgggtgctggtaggcggagtccttgcagctttggccgcatactgcctgacgacaggcagtgtggtcattgtgggcaggatcatcttgtccgggaagccagctgtcgttcccgacagggaagtcctctaccaggagttcgatgagatggaagagtgtgcctcacaacttccttacatcgagcagggaatgcagctcgccgagcaattcaagcaaaaggcgctcgggttgttgcaaacggccaccaagcaagcggaggctgctgctcccgtggtggagtccaagtggcgagcccttgagaccttctgggcgaagcacatgtggaatttcatcagcggaatacagtacctagcaggcttatccactctgcctggaaaccccgcgatagcatcattgatggcatttacagcttctatcactagcccgctcaccacccaaaacaccctcctgtttaacatcttggggggatgggtggctgcccaactcgctcctcccagcgctgcgtcagctttcgtgggcgccggcatcgccggagcggctgttggcagcataggccttgggaaggtgctcgtggacatcttggcgggctatggggcaggggtagccggcgcactcgtggcctttaaggtcatgagcggcgaggtgccctccaccgaggacctggtcaacttactccctgccatcctctctcctggtgccctggtcgtcggggtcgtgtgcgcagcaatactgcgtcggcacgtgggcccgggagagggggctgtgcagtggatgaaccggctgatagcgttcgcttcgcggggtaaccacgtctcccctacgcactatgtgcctgagagcgacgctgcagcacgtgtcactcagatcctctctagccttaccatcactcaactgctgaagcggctccaccagtggattaatgaggactgctctacgccatgctccggctcgtggctaagggatgtttgggattggatatgcacggtgttgactgacttcaagacctggctccagtccaaactcctgccgcggttaccgggagtccctttcctgtcatgccaacgcgggtacaagggagtctggcggggggacggcatcatgcaaaccacctgcccatgcggagcacagatcgccggacatgtcaaaaacggttccatgaggatcgtagggcctagaacctgcagcaacacgtggcacggaacgttccccatcaacgcatacaccacgggaccttgcacaccctccccggcgcccaactattccagggcgctatggcgggtggctgctgaggagtacgtggaggttacgcgtgtgggggatttccactacgtgacgggcatgaccactgacaacgtaaagtgcccatgccaggttccggcccccgaattcttcacggaggtggatggagtgcggttgcacaggtacgctccggcgtgcaaacctcttctacgggaggacgtcacgttccaggtcgggctcaaccaatacttggtcgggtcgcagctcccatgcgagcccgaaccggacgtaacagtgcttacttccatgctcaccgatccctcccacattacagcagagacggctaagcgtaggctggctagagggtctcccccctctttagccagctcatcagctagccagttgtctgcgccttctttgaaggcgacatgcactacccaccatgactccccggacgctgacctcatcgaggccaacctcttgtggcggcaggagatgggcggaaacatcactcgcgtggagtcagagaataaggtagtaattctggactctttcgaaccgcttcacgcggagggggatgagagggagatatccgtcgcggcggagatcctgcgaaaatccaggaagttcccctcagcgttgcccatatgggcacgcccggactacaatcctccactgctagagtcctggaaggacccggactacgtccctccggtggtacacggatgcccattgccacctaccaaggctcctccaataccacctccacggagaaagaggacggttgtcctgacagaatccaatgtgtcttctgccttggcggagctcgccactaagaccttcggtagctccggatcgtcggccgttgatagcggcacggcgaccgcccttcctgacctggcctccgacgacggtgacaaaggatccgacgttgagtcgtactcctccatgcccccccttgaaggggagccgggggaccccgatctcagcgacgggtcttggtctaccgtgagtgaggaggctagtgaggatgtcgtctgctgctcaatgtcctatacgtggacaggcgccctgatcacgccatgcgctgcggaggaaagtaagctgcccatcaacccgttgagcaactctttgctgcgtcaccacaacatggtctacgccacaacatcccgcagcgcaagcctccggcagaagaaggtcacctttgacagattgcaagtcctggatgatcattaccgggacgtactcaaggagatgaaggcgaaggcgtccacagttaaggctaagcttctatctatagaggaggcctgcaagctgacgcccccacattcggccaaatccaaatttggctatggggcaaaggacgtccggaacctatccagcagggccgttaaccacatccgctccgtgtgggaggacttgctggaagacactgaaacaccaattgacaccaccatcatggcaaaaagtgaggttttctgcgtccaaccagagaagggaggccgcaagccagctcgccttatcgtattcccagacctgggagttcgtgtatgcgagaagatggccctttacgacgtggtctccacccttcctcaggccgtgatgggctcctcatacggatttcaatactcccccaagcagcgggtcgagttcctggtgaatacctggaaatcaaagaaatgccctatgggcttctcatatgacacccgctgttttgactcaacggtcactgagagtgacattcgtgttgaggagtcaatttaccaatgttgtgacttggcccccgaggccagacaggccataaggtcgctcacagagcggctttacatcgggggtcccctgactaactcaaaagggcagaactgcggttatcgccggtgccgcgcaagtggcgtgctgacgactagctgcggtaataccctcacatgttacttgaaggccactgcagcctgtcgagctgcaaagctccaggactgcacgatgctcgtgaacggagacgaccttgtcgttatctgtgaaagcgcgggaacccaggaggatgcggcggccctacgagccttcacggaggctatgactaggtattccgccccccccggggatccgccccaaccagaatacgacctggagctgataacatcatgttcctccaatgtgtcagtcgcgcacgatgcatctggcaaaagggtatactacctcacccgtgaccccaccaccccccttgcacgggctgcgtgggagacagctagacacactccaatcaactcttggctaggcaatatcatcatgtatgcgcccaccctatgggcaaggatgattctgatgactcactttttctccatccttctagctcaagagcaacttgaaaaagccctggattgtcagatctacggggcttgctactccattgagccacttgacctacctcagatcattgaacgactccatggtcttagcgcatttacactccacagttactctccaggtgagatcaatagggtggcttcatgcctcaggaaacttggggtaccacccttgcgaacctggagacatcgggccagaagtgtccgcgctaagctactgtcccagggggggagggccgccacttgtggcagatacctctttaactgggcagtaaggaccaagcttaaactcactccaatcccggccgcgtcccagctggacttgtctggctggttcgtcgctggttacagcgggggagacatatatcacagcctgtctcgtgcccgaccccgctggtttccgttgtgcctactcctactttctgtaggggtaggcatttacctgctccccaaccgatgaacggggagctaaccactccaggccttaagccatttcctgttttttttttttttttttttttttttttctttttttttttctttcctttccttctttttttcctttctttttcccttctttaatggtggctccatcttagccctagtcacggctagctgtgaaaggtccgtgagccgcatgactgcagagagtgctgatactggcctctctgcagatcatgt

SEQ ID NO: 20 - DNA, 576 bp, Hepatitis C virus
atgagcaccaaccccaagccccagcgcaagaccaagcggaacaccaaccggagaccccaggacgtcaagttcccaggaggaggccagatcgtgggcggcgtgtacctgctgccccgccgggggccccggctgggcgtgcgcgccacccgcaagaccagcgagcgctcccagccaagaggcagacgccagccgatcccgaaggcccgccgccctgagggccgggcttgggcccagccaggctacccctggcccctgtatggcaacgagggcctgggatgggctgggtggctcctcagcccccgggggtctaggcccagttggggaccgaccgacccccgcaggcgcagccgcaacctgggaaaggtgatcgacacgctcacctgcggcttcgccgacttgatgggatacatccctctggtgggggcccctctgggcggagccgcgcgcgccctggctcacggggtccgggtgctcgaggacggggtgaactacgccaccgggaacctgcccggctgcagcttctccatcttcctgctggcgctgctgagctgcctcaccatccccgctagcgcatga

SEQ ID NO: 21 - DNA, 1899 bp, Hepatitis C virus
atggcccccatcaccgcctacagccagcagacccggggactgctcggctgcatcatcacctctctgacaggccgggataagaaccaggtggagggcgaggtgcaggtcgtctcgaccgctacccaaagcttcctggccacctgtatcaacggagtctgctggacggtgtaccatggcgccggcagcaagaccctcgccgggcctaagggccccatcacccagatgtacaccaacgtggaccaggacctggtgggctggcaggcgccccccggggcgaggagtatgaccccatgcacctgcgggagctctgacctgtatctggtgaccagacatgccgatgtcatcccggtgaggcgtcgcggggacagtagagggagcctgctgagcccccgccccgtcagctacctgaaggggtccgtgggcggccccctgctgtgcccctctggccacgtggtcggcatcttcagggccgccgtgtgcacgcgcggcgtggccaaggccgtggactttatccccgtggagagcatggagaccaccatgcgctcccccgtgttcaccgacaacagcagcccccccgccgtgcctcagaccttccaggtcgcccacctccatgctccgacgggctccgggaagtccacgaaggtgcccgccgcgtacgcggcccagggatacaaggtgctggtcctcaaccctagcgtggctgccacactcgggtttggagcgtacatgagcaaggcgcacggcatcgaccccaacatcagaactggcgtccggaccatcacaaccggcgctcccatcacttactctacctacggcaagttcctggctgatggggggtgtagtgggggcgcgtacgatattatcatctgccaggagtgccactctaccgacagcaccacaatcctgggcatcggcaccgtcctcgaccaggctgagacagcgggcgcccgcctggtggtgctggccacggccactccccccggctccgtcacggtgccccaccccaatatcgaggaggtggccctgagcaacaacggcgagatcccattctacggcaaggctatcccgatcgaggcgattaagggaggcagacatctgatcttctgccacagcaagaagaagtgcgacgagctcgccgccaagctgagcggcctcggactcaacgccgtggcttactacaggggactggacgtgtccgtgatcccgaccagcggagacgtggtggtcgtggccaccgacgccctgatgaccggcttcaccggagacttcgacagcgtcatcgactgcaacacctgcgtgacccagaccgtggacttcagcctggaccccaccttcaccatcgagaccaccacagtgccccaggacgccgtgtcccgcagccagcgccggggccggaccggccgcggccggagtggcatctataggttcgtgaccccgggcgagcgccccagcggcatgttcgatagttccgtgctgtgcgagtgctacgacgccggatgcgcgtggtacgagctgaccccggcggagacctctgtccgcctgagggcttacttgaataccccgggcctgcccgtgtgccaggatcatctcgagttctgggaatccgtcttcaccggcctgacacacatcgacgcccatttcttgtcccaaaccaagcaggctggcgacaatttcccgtatctggtcgcgtaccaggccacggtgtgcgcgcgtgcgcaggctcccccccctagctgggatcagatgtggaagtgcctgatccgcctgaagcccaccctgcatgggcccacccccctgctgtaccgcctgggcgcggtgcagaacgaagtcaccttgacccaccccatcaccaagtacatcatggcgtgcatgtccgctgacctggaggtggtcacctga

SEQ ID NO: 22 - DNA, 645 bp, Hepatitis C virus
atgttttgggccaagcatatgtggaacttcatcagcggcatccagtacctcgccgggctgagcaccctcccgggcaaccccgcgatcgcaagcctgatggcgttcacagcgagcatcacctcccccctgactacccagaacacactgctgttcaacatcctggggggctgggtcgccgctcagctggcccctccttccgccgccagcgcctttgtgggggcgggaatcgccggggccgccgtcggctccatcggactgggcaaggtgctggtcgacatcctggcgggctacggcgcgggagtcgccggagccctggtggccttcaaggtgatgagcggagaggtgccaagcactgaggacctggtgaacctgctgccggcgatcctgagcccgggcgccctggtggtgggcgtggtgtgtgctgccatcctcaggcgccacgtgggcccgggcgagggagccgtgcagtggatgaaccgcctgatcgcctttgcctcccgcggcaaccacgtcagccctacacattacgtgcccgagagcgatgccgccgcccgcgtgacccagatcctgagctccctgaccatcacccagctgctcaagaggctgcaccagtggatcaacgaggactgctccaccccttgctga

SEQ ID NO: 23 - DNA, 1779 bp, Hepatitis C virus
atgtccatgtcctacacctggaccggcgccctgatcaccccctgcgccgccgaggagagcaagctcccgattaaccccctgtccaactctctgctccgccatcacaacatggtgtatgccaccacctcccgctctgcgagcctccgccagaagaaggtgacgttcgacagactgcaggtgctggacgaccattacagggacgtgctgaaggaaatgaaggccaaggctagcaccgtgaaggccaagctgctcagcattgaggaggcttgcaagctgacccccccccacagtgctaaatccaagttcggctacggcgccaaggacgtgaggaacctgtcctcgcgcgctgtgaaccatatccgcagcgtgtgggaggacctgctcgaggacaccgagacccccatcgacacaaccatcatggccaagtccgaggtgttctgcgtgcagccggagaaaggaggccgcaagccagcccgcctgatcgtcttccccgacctgggcgtgagagtctgcgagaagatggccctctacgacgtggtgtccaccctgccgcaggccgtgatggggagttcctacggcttccagtacagcccgaagcagagggtggagttcctggtgaacacgtggaagtctaagaaatgccccatggggttcagttacggaacaaggtgcttcgggagtactgtgaccgaatccgatatccgcgtggaggagagcatctaccagtgttgtgacctcgcccccgaggcgagacaggccatccgctccctgaccgagaggctgtatatcggcggcccactgaccaacagcaaggggcagaactgcggctatcgccgttgtcgggcctccggggtgctcaccacctcttgcgggaacaccctcacctgctacctcaaggcgaccgctgcctgcagagccgcgaagctgcaggactgcaccatgctcgtgaacggcgacgatctggtggtgatctgtgagtccgcgggcacgcaggaggacgcggcggccctgcgggcgttcacagaggccatgacacgctacagtgccccccccggcgaccccccccagcccgaatacgatctggagctcatcactagttgcagctcgaacgtgtctgtggcccatgacgcttctggcaaacgggtgtattatctgacgcgcgatcccaccacccccctcgccagagccgcgtgggagacagctcggcacacccctgtgaactcttggctgggcaacatcatcatgtacgcccctaccctgtgggctcgcatgatcctgatgacccacttcttcagtatcctcctcgctcaggagcagctggagaaggcgctcgactgccagatctacggcgcctgctatagtatcgagcctctcgacctgccccagatcatcgagagactgcatgggctcagcgccttctccctccatagttactctcctggagaaattaaccgggtggcgagctgtctgcggaagctcggcgtcccccctctgcgcgtttggcggcatcgcgccaggagtgtgagggccaagctgctgagccagggcggaagggccgccacctgcggccggtatctcttcaactgggccgtgcgcaccaagctcaagctcacccccatccctgccgccagtcagctggatctcagtgggtggttcgtggccggctattctggcggcgacatctaccactccctcagcagggcgcgcccccgctggttccccctgtgcctgctgctcctgagcgtcggagtcggcatctacctgctgcccaaccgctga

SEQ ID NO: 24 - protein, 3010 aa, Hepatitis C virus
MSTNPKPQRKTKRNTNRRPQDVKFPGGGQIVGGVYLLPRRGPRLGVRATRKASERSQPRGRRQPIPKARRPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGIYHVTNDCSNSSIVYEAADVIMHTPGCVPCVQEGNSSRCWVALTPTLAARNASVPTTTIRRHVDLLVGTAAFCSAMYVGDLCGSIFLVSQLFTFSPRRHETVQDCNCSIYPGHVSGHRMAWDMMMNWSPTTALVVSQLLRIPQAVVDMVAGAHWGVLAGLAYYSMVGNWAKVLIVALLFAGVDGETHTTGRVAGHTTSGFTSLFSSGASQKIQLVNTNGSWHINRTALNCNDSLQTGFFAALFYAHKFNSSGCPERMASCRPIDWFAQGWGPITYTKPNSSDQRPYCWHYAPRPCGVVPASQVCGPVYCFTPSPVVVGTTDRSGVPTYSWGENETDVMLLNNTRPPQGNWFGCTWMNSTGFTKTCGGPPCNIGGVGNRTLICPTDCFRKHPEATYTKCGSGPWLTPRCLVDYPYRLWHYPCTLNFSIFKVRMYVGGVEHRLNAACNWTRGERCNLEDRDRSELSPLLLSTTEWQILPCAFTTLPALSTGLIHLHQNIVDVQYLYGVGSAFVSFAIKWEYILLLFLLLADARVCACLWMMLLIAQAEAALENLVVLNAASVAGAHGILSFLVFFCAAWYIKGRLAPGAAYAFYGVWPLLLLLLALPPRAYALDREMAASCGGAVLVGLVFLTLSPYYKVFLTRLIWWLQYFITRAEAHMQVWVPPLNVRGGRDAIILLTCAVHPELIFDITKLLLAILGPLMVLQAGITRVPYFVRAQGLIRACMLVRKVAGGHYVQMVFMKLGALTGTYVYNHLTPLRDWAHAGLRDLAVAVEPVVFSAMETKVITWGADTAACGDIILGLPVSARRGKEIFLGPADSLEGQGWRLLAPITAYSQQTRGVLGCIITSLTGRDKNQVEGEVQVVSTATQSFLATCINGVCWTVYHGAGSKTLAGPKGPITQMYTNVDLDLVGWQAPPGARSMTPCSCGSSDLYLVTRHADVIPVRRRGDSRGSLLSPRPVSYLKGSSGGPLLCPSGHVVGVFRAAVCTRGVAKAVDFIPVESMETTMRSPVFTDNSTPPAVPQTFQVAHLHAPTGSGKSTKVPAAYAAQGYKVLVLNPSVAATLGFGAYMSKAHGIDPNIRTGVRTITTGGSITYSTYGKFLADGGCSGGAYDIIICDECHSTDSTTILGIGTVLDQAETAGARLVVLATATPPGSVTVPHPNIEEIGLSNNGEIPFYGKAIPIEAIKGGRHLIFCHSKKKCDELAAKLTGLGLNAVAYYRGLDVSVIPPIGDVVVVATDALMTGFTGDFDSVIDCNTCVTQTVDFSLDPTFTIETTTVPQDAVSRSQRRGRTGRGRSGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVILTHPITKYIMACMSADLEVVTSTWVLVGGVLAALAAYCLTTGSVVIVGRIILSGKPAVVPDREVLYQEFDEMEECASQLPYIEQGMQLAEQFKQKALGLLQTATKQAEAAAPVVESKWRALETFWAKHMWNFISGIQYLAGLSTLPGNPAIASLMAFTASITSPLTTQNTLLFNILGGWVAAQLAPPSAASAFVGAGIAGAAVGSIGLGKVLVDILAGYGAGVAGALVAFKVMSGEVPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKTWLQSKLLPRLPGVPFLSCQRGYKGVWRGDGIMQTTCPCGAQIAGHVKNGSMRIVGPRTCSNTWHGTFPINAYTTGPCTPSPAPNYSRALWRVAAEEYVEVTRVGDFHYVTGMTTDNVKCPCQVPAPEFFTEVDGVRLHRYAPACKPLLREDVTFQVGLNQYLVGSQLPCEPEPDVTVLTSMLTDPSHITAETAKRRLARGSPPSLASSSASQLSAPSLKATCTTHHDSPDADLIEANLLWRQEMGGNITRVESENKVVILDSFEPLHAEGDEREISVAAEILRKSRKFPSALPIWARPDYNPPLLESWKDPDYVPPVVHGCPLPPTKAPPIPPPRRKRTVVLTESNVSSALAELATKTFGSSGSSAVDSGTATALPDLASDDGDKGSDVESYSSMPPLEGEPGDPDLSDGSWSTVSEEASEDVVCCSMSYTWTGALITPCAAEESKLPINPLSNSLLRHHNMVYATTSRSASLRQKKVTFDRLQVLDDHYRDVLKEMKAKASTVKAKLLSIEEACKLTPPHSAKSKFGYGAKDVRNLSSRAVNHIRSVWEDLLEDTETPIDTTIMAKSEVFCVQPEKGGRKPARLIVFPDLGVRVCEKMALYDVVSTLPQAVMGSSYGFQYSPKQRVEFLVNTWKSKKCPMGFSYDTRCFDSTVTESDIRVEESIYQCCDLAPEARQAIRSLTERLYIGGPLTNSKGQNCGYRRCRASGVLTTSCGNTLTCYLKATAACRAAKLQDCTMLVNGDDLVVICESAGTQEDAAALRAFTEAMTRYSAPPGDPPQPEYDLELITSCSSNVSVAHDASGKRVYYLTRDPTTPLARAAWETARHTPINSWLGNIIMYAPTLWARMILMTHFFSILLAQEQLEKALDCQIYGACYSIEPLDLPQIIERLHGLSAFTLHSYSPGEINRVASCLRKLGVPPLRTWRHRARSVRAKLLSQGGRAATCGRYLFNWAVRTKLKLTPIPAASQLDLSGWFVAGYSGGDIYHSLSRARPRWFPLCLLLLSVGVGIYLLPNR

(19) United States
(12) Patent Application Publication
(10) Pub. No.: US 2009/0104231 A1
(43) Pub. Date: Apr. 23, 2009

(54) VACCINE

(75) Inventors: Sara Brett, Stevenage (GB); Paul Andrew Hamblin, Stevenage (GB); Louis Ogilvie, Stevenage (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 12/174,715

(22) Filed: Jul. 17, 2008

Related U.S. Application Data
(63) Continuation of application No. 10/535,047, filed on Jan. 5, 2006, now abandoned.

(30) Foreign Application Priority Data
Nov. 15, 2002 (GB) 0226722.7

(51) Int. Cl. A61K 39/00

(52) U.S. Cl. 424/228.1

(57) ABSTRACT

The present invention relates to methods and compositions useful in the treatment and prevention of Hepatitis C virus (HCV) infections and the symptoms and diseases associated therewith. In particular the present invention relates to DNA vaccines comprising polynucleotide sequences encoding HCV proteins, and methods of treatment of individuals infected with HCV comprising administration of the vaccines of the present invention.

The present invention relates to methods and compositions useful in the treatment and prevention of Hepatitis C virus (HCV) infections and the symptoms and diseases associated therewith. In particular the present invention relates to DNA vaccines comprising polynucleotide sequences encoding HCV proteins, and methods of treatment of individuals infected with HCV comprising administration of the vaccines of the present invention.

### BRIEF DESCRIPTION OF FIGURES

FIG. 1—Nucleotide Sequence of HCV J4L6 genome wild-type cDNA sequence, reference accession number AF054247,

FIG. 2—Nucleotide sequence of codon optimised HCV Core polynucleotide.

FIG. 3—Nucleotide sequence of codon optimised HCV NS3 polynucleotide.

FIG. 4—Nucleotide sequence of codon optimised HCV NS4B polynucleotide.

FIG. 5—Nucleotide sequence of codon optimised HCV NS5B polynucleotide.

FIG. 6—Protein sequence of HCV J4L6 genome (wild-type sequence).

FIG. 7—Mammalian expression vector p7313-ie showing Not I and BamHI unique cloning sites

FIG. 8—Immune responses to Core in C57BL mice.

FIG. 9—NS3 immunogenicity (wild type and codon optimised).

FIG. 10—Immune responses to NS4B.

FIG. 11—NS5B immune responses.

FIG. 12—Expression of HCV antigens.

FIG. 13—(A) NS3 protein ELISPOT assay with rat anti mouse IL-2—(B)—NS3 protein ELISPOT assay with rat anti mouse IFNg.

FIG. 14—Vaccinia ELISPOT assay with rat anti mouse IL-2 and rat anti mouse IFNg.

FIG. 15—PMID immunisation of C57BL mice with HCV polyproteins.

FIG. 16—Western blot analysis of HCV antigens in dual promoter constructs.

FIG. 17—Comparison of NS3 T cell response induced by dual promoter constructs.

FIG. 18—DNA agarose gel showing range of genes encoding fragments of Core.

FIG. 19—Effect of expression of range of genes encoding fragments of Core on the expression level of NS4B5B fusion by co-tranfection in 293T cells.

FIG. 20—Effect of Core and Core-151 upon expression of NS3, NS5B, NS4B5B and NS34B5B after co-transfection in 293 T cells.

FIG. 21—Effect on expression of fusion proteins, after substitution of Core-151 for Core-191 in transient transfection in 293T cells.

FIG. 22—Comparison of effect of Core 191 and Core 151 on immune responses to NS3 using double fusion constructs.

FIG. 23—Comparison of effect of Core 191 and Core 151 on immune responses to NS3 using triple fusion constructs.

HCV was identified recently as the leading causative agent of post-transfusion and community acquired non A, non B hepatitis. Approximately 170 m people are chronically infected with HCV, with prevalence between 1-10%. The health care cost in the US, where the prevalence is 1.8%, is estimated to be $2 billion. Between 40-60% of liver disease is due to HCV and 30% UK transplants are for HCV infections. Although HCV is initially a sub-clinical infection more than 90% of patients develop chronic disease. The disease process typically develops from chronic active hepatitis (70%), fibrosis, cirrhosis (40%) to hepato-cellular carcinoma (60%). Infection to cirrhosis has a median time of 20 years and that for hepato-cellular carcinoma of 20 years (Lauer G. and Walker B. 2001, Hepatits C virus Infection. N Engl J. Med 345, 41, Cohen J. 2001. The Scientific challenge of Hepatitis C. Science 285 (5424) 26.

There is a great need for the improved treatment of HCV. There are currently no small molecule replication inhibitors available. The current gold standard of ribovirin and PEGylated interferon represents the mainstay for treating HCV infection. However the ability of the current regimens to achieve sustained response remains sub-optimal (overall 50% response rate for up to 6 months, however, for genotype 1b the response rate is lower (27%). This treatment is also associated with unpleasant side effects. This results in high fall out rate, especially after first 6 months of treatment.

Several studies have shown that the individual HCV proteins are immunogenic in normal mice, including following immunisation with DNA. Several HCV vaccines are currently in clinical trial for either prophylaxis or therapy. The most advanced are currently in Phase 2 by Chiron and Innogenetics using E1 or E2 envelope proteins. An epitope vaccine by Transvax is also in Phase 2. Several vaccines are in preclinical development which use sequences from core and non-structural antigens using a variety of delivery systems including DNA.

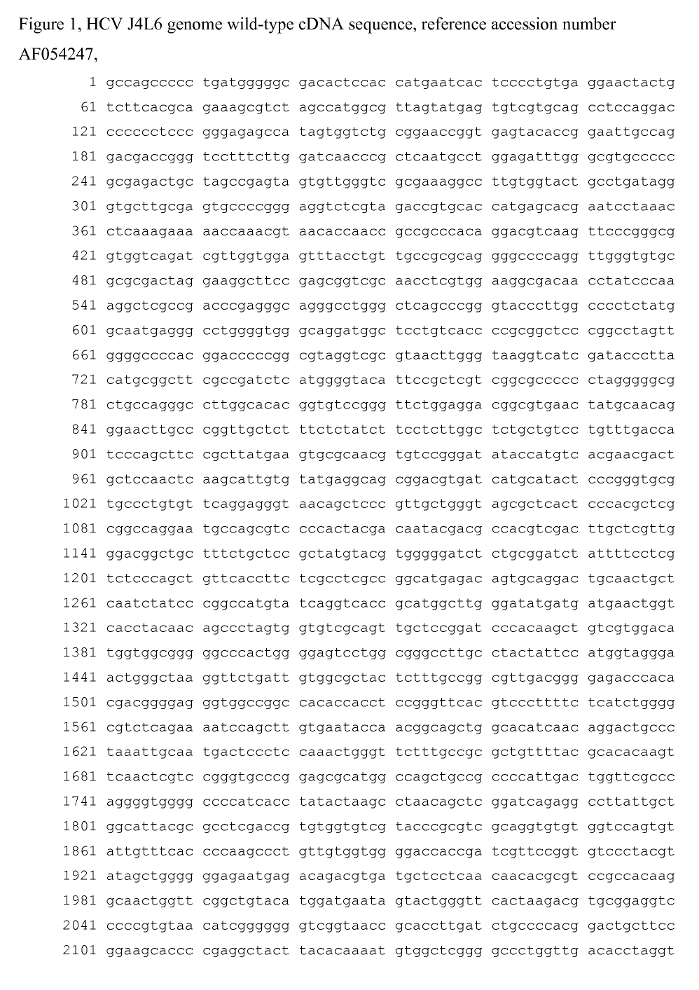

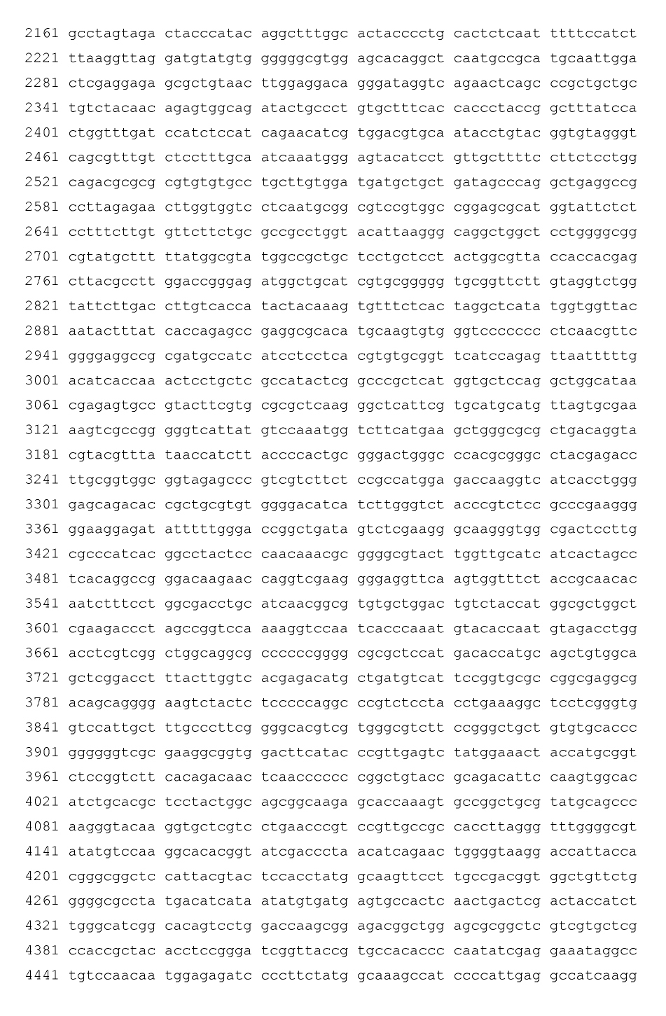

HCV is a positive strand RNA virus of the flaviviradae family, whose genome is 9.4 kb in length, with one open reading frame. The HCV genome is translated as a single polyprotein, which is then processed by host and viral proteases to produce structural proteins (core, envelope E1 and E2, and p7) and six non-structural proteins with various enzymatic activities. The genome of the HCV J4L6 isolate, which is an example of the 1b genotype, is found as accession number AF054247 (Yanagi, M., St Claire, M., Shapiro, M., Emerson, S. U., Purcell, R. H. and Bukh, J. “Transcripts of a chimeric cDNA clone of hepatitis C virus genotype 1b are infectious in vivo”. Virology 244 (1), 161-172 (1998)), and is shown in FIG. 1.

The envelope proteins are responsible for recognition, binding and entry of virus onto target cells. The major non-structural proteins involved in viral replication include NS2 (Zn dependent metaloproteinase), NS3 (serine protease/helicase), NS4A (protease co-factor), NS5A and NS5B (RNA polymerase) (Bartenschlager B and Lohmann V. 2000. Replication of hepatitis C virus. J. Gen Virol 81, 1631).

The structure of the HCV polyprotein can be represented as follows (the figures refer to the position of the first amino acid of each protein; the full polyprotein of the J4L6 isolate is 3010 amino acids in length)

| Core | E1 | E2 | P7 | NS2 | NS3 | NS4A | NS4B | NS5A | NS5B |
|---|---|---|---|---|---|---|---|---|---|
| 1-191 | | | | | 1027-1657 | | 1712-1972 | | 2420-3010 |

The virus has a high mutation rate and at least six major genotypes have been defined based in the nucleotide sequence of conserved and non-conserved regions. However there is additional heterogeneity as HCV isolated from a single patient is always presented as a mixture of closely related genomes or quasi-species.

The HCV genome shows a high degree of genetic variation, which has been classified into 6 major genotypes (1a, 1b, 2, 3, 4, 5, and 6). Genotypes 1a, 1b, 2 and 3 are the most prevalent in Europe, North and South America, Asia, China, Japan and Australia. Genotypes 4 and 5 are predominant in Africa and genotype 6 S.E Asia.

There is a great need, therefore, for improved treatments of HCV infection and also to provide treatments that are diverse in the ability to treat a number of HCV genotypes. In a first aspect of the present invention there is provided novel vaccine formulations that are diverse in their protection against various genotypes.

HCV vaccines comprising polynucleotides encoding one or more HCV proteins have been described. Vaccines comprising plasmid DNA or Semliki Forest Virus vectors encoding NS3 were described by Brinster et al. (2002, Journal of General Virology, 83, 369-381). Polynucleotide vaccines encoding NS5B are disclosed in WO 99/51781. Codon optimised genes, and vaccines comprising them, encoding HCV E1, E1+E2 fusions, NS5A and NS5B proteins are described in WO 97/47358. WO 01/04149 discloses polypeptides or polynucleotides encoding mosaics of HCV epitopes, derived from within Core, NS3, NS4 or NS5A. Fusion proteins, and DNA encoding such fusion proteins, comprising NS3, NS4, NS5A and NS5B, that are useful in vaccines are described in WO 01/30812; optionally the fusion proteins are said to comprise fragments of the Core protein. WO 03/031588 describes an adenovirus vector, that is suitable for use as a vaccine, which encodes the HCV proteins NS3-NS4A-NS4B-NS5A-NS5B.

Vaccines comprising polypeptides comprising “unprocessed” core protein and a non-structural protein are described in WO 96/37606.

The present invention relates to the provision of a polynucleotide vaccine that encodes the HCV proteins Core, NS3, NS4B and NS5B. The polynucleotide vaccines of the present invention do not encode the NS4A HCV protein and/or the NS5A protein. Preferably, the polynucleotide vaccines of the present invention encode Core, NS3, NS4B and NS5B HCV proteins, and no other HCV proteins. The present invention also provides the use of a polynucleotide vaccine encoding these antigens in medicine, and in the manufacture of a medicament for the treatment, or prevention, of an HCV infection.

The polynucleotide sequences used in the vaccines of the present invention are preferably DNA sequences.

The polynucleotides encoding the HCV proteins may be in many combinations or configurations. For example, the proteins may be expressed as individual proteins, or as fusion proteins. An example of a fusion, which could either be at the DNA or protein level, would be a double fusion which consists of a single polypeptide or polynucleotide containing or encoding the amino acid sequences of NS4B and NS5B (NS4B-NS5B), a triple fusion containing or encoding the amino acid sequences of NS3-NS4B-NS5B, or a fusion of all four antigens of the present invention (Core-NS3-NS4B-NS5B).

Preferred fusions of the present invention are polynucleotides that encode the double fusion between NS4B and NS5B (NS4B-NS5B or NS5B-NS4B); and between Core and NS3 (NS3-Core or Core-NS3). Preferred triple fusions are polynucleotides that encode the amino acid sequences of NS3-NS4B-NS5B.

The polynucleotides of the present invention encoding the single antigens or fusion proteins could be present in a single, or in multiple expression vectors. Preferably the polynucleotides encoding each antigen are present in the same expression vector or plasmid. In this context the polynucleotides encoding the HCV proteins may be in a single expression cassette, or in multiple in series expression cassettes.

In order to optimise the expression of the other HCV proteins, the polynucleotide encoding the HCV Core protein is preferably present in an expression cassette that is downstream of an expression cassette that contains the polynucleotide that encodes at least one of the other HCV proteins. Preferably the HCV Core protein is preferably present in an expression cassette that is downstream of an expression cassette that contains the polynucleotide that encodes NS5B.

The polypeptides encoded by the oligonucleotide vaccines of the present invention may comprise the full length amino acid sequence or alternatively the polypeptides may be shorter than the full length proteins, in that they comprise a sufficient proportion of the full length polynucleotide sequence to enable the expression product of the shortened gene to generate an immune response which cross reacts with the full length protein. For example, a polynucleotide of the invention may encode a fragment of a HCV protein which is a truncated HCV protein in which regions of the original sequence have been deleted, the final fragment comprising less than 90% of the original full length amino acid sequence, and may be less than 70% or less than 50% of the original sequence. Alternatively speaking, a polynucleotide which encodes a fragment of at least 8, for example 8-10 amino acids or up to 20, 50, 60, 70, 80, 100, 150 or 200 amino acids in length is considered to fall within the scope of the invention as long as the encoded oligo or polypeptide demonstrates HCV antigenicity. In particular, but not exclusively, this aspect of the invention encompasses the situation when the polynucleotide encodes a fragment of a complete HCV protein sequence and may represent one or more discrete epitopes of that protein.

In preferred vaccines of the present invention at least one, and preferably all, of the HCV polypeptides are inactivated by truncation or mutation. For example the helicase and protease activity of NS3 is preferably reduced or abolished by mutation of the gene. Preferably NS5B polymerase activity of the expressed polypeptide is reduced or abolished by mutation. Preferably NS4B activity of the expressed polypeptide is reduced or abolished by mutation. Preferably activity of the Core protein of the expressed polypeptide is reduced or abolished by truncation or mutation. Mutation in this sense could comprise an addition, deletion, substitution or rearrangement event to polynucleotide encoding the polypeptide. Alternatively the full length sequence may be expressed in two or more separate parts.

The functional structure and enzymatic function of the HCV polypeptides NS3 and NS5B are described in the art.

NS5B has been described as an RNA-dependent RNA polymerase Qin et al., 2001, Hepatology, 33, pp 728-737; Lohmann et al., 2000, Journal of Viral Hepatitis; Lohmann et al., 1997, November, Journal of Virology, 8416-8428; De Francesco et al., 2000, Seminars in Liver Disease, 20(1), 69-83. The NS5B polypeptide has been described as having four functional motifs A, B, C and D.

Preferably the NS5B polypeptide sequence encoded by polynucleotide vaccines of the present invention is mutated to reduce or remove RNA-dependent RNA polymerase activity. Preferably the polypeptide is mutated to disrupt motif A of NS5B, for example a substitution of the Aspartic acid (D) in position 2639 to Glycine (G); or a substitution of Aspartic acid (D) 2644 to Glycine (G). Preferably, the NS5B polypeptide encoded by the vaccine polynucleotide contains both of these Aspartic acid mutations.

Preferably, the encoded NS5B contains a disruption in its motif C. For example, Mutation of D₂₇₃₇, an invariant aspartic acid residue, to H, N or E leads to the complete inactivation of NS5B.

Preferably the NS5B encoded by the DNA vaccines of the present invention comprise a motif A mutation, which may optionally comprise a motif C mutation. Preferred mutations in motif A include Aspartic acid (D) 2639 to Glycine and aspartic acid (D) 2644 Glycine. Preferably both mutations are present. Additional further consensus mutations may be present, as set forth below in example 1.

NS3 has been described as having both protease and helicase activity. The NS3 polypeptides encoded by the DNA vaccines of the present invention are preferably mutated to disrupt both the protease and helicase activities of NS3. It is known that the protease activity of NS3 is linked to the “catalytic triad” of H-1083, D-1107 and S-1165. Preferably the NS3 encoded by the vaccines of the present invention comprises a mutation in the Catalytic triad residues, and most preferably the NS3 comprises single point mutation of Serine 1165 to valine (De Francesco, R., Pessi, a and Steinkuhler C. 1998. The hepatitis C Virus NS3 proteinase: structure and function of a zinc containing proteinase. Anti-Viral Therapy 3, 1-18.).

The structure and function of NS3 can be represented as:

Protease
Catalytic triad:
H-1083
D-1107
S-1165
Helicase
Established functional motifs:

| I | II | III | IV |
|---|---|---|---|
| GKS | DECH | TAT | QRrGRtGR |

Four critical motifs for the helicase activity of NS3 have been identified, I, II, III and IV. Preferably the NS3 encoded by the DNA vaccines of the present invention comprise disruptive mutations to at least one of these motifs. Most preferably, there is a substitution of the Aspartic acid 1316 to glutamine (Paolini, C, Lahm A, De Francesco R and Gallinari P 2000, Mutational analysis of hepatitis C virus NS3-associated helicase. J. Gen Virol. 81, 1649). Neither of these most preferred NS3 mutations, S165V or D11316Q, lie within known or predicted T cell epitopes.

Most preferably the NS3 polypeptide encoded by the DNA vaccines of the present invention comprise Serine (S) 1165 to Valine (V) and an Aspartic acid (D) 1316 to Glutamine (Q) mutation. Additionally one or more of the consensus mutations as set forth in example 1 may be present.

The biological functions of HCV core protein are complex and do not correlate with discrete point mutations (McLauchlan J. 2000. Properties of the hepatitis C virus core protein: a structural protein that modulates cellular processes. J of Viral Hepatitis 7, 2-4). There is evidence that core directly interacts with the lymphotoxin β receptor, and can also interfere with NFκB and PKR pathways and can influence cell survival and apoptosis. A recombinant vaccinia construct expressing core was found to inhibit cellular responses to vaccinia making it more virulent in vivo.

During an infection, the Core protein is cleaved at two sites from the viral polyprotein by host cell proteases. The first cleavage is at 191 which generates the N-terminal end of E1. The residue at which the second cleavage takes place has not been precisely located and lies between amino acids 174 and 191, thereby liberating a short Core peptide sequence of approximately 17 amino acids in length (McLauchlan J. (2000) J. Viral Hepatitis. 7, 2-14; YasuiK, Lau J Y N, Mizokami M., et al., J. Virol 1998. 72 6048-6055).

The Core polypeptides used in the vaccines of the present invention are either full length or in a truncated form. The Core polypeptide may be full length, but the sequence of which is rearranged to abrogate any activity of Core protein. The Core polypeptide may be split into at least two fragments, and most preferably forming a polypeptide consisting of Core amino acids 66-191 followed onto amino acids 1-65, and alternatively Core amino acids 105-191 followed by Core amino acids 1-104.

Most preferably, in order to minimise the negative effect of Core upon the production of other HCV proteins in the same cell, the Core protein used is a truncated protein. In a preferred aspect of the present invention the Core protein that is encoded is truncated from the carboxy terminal end in a sufficient amount to reduce the inhibitory effect of Core upon the expression of other HCV proteins. Most preferably the Core protein is truncated from the carboxy terminal end, such that the sequence of the protein produced lacks the naturally liberated C-terminal peptide sequence arising from the second cleavage of Core; more preferably the protein lacks at least the last 10 amino acids, preferably lacks at least the last 15 amino acids, more preferably lacks the last 20 amino acids, more preferably lacks the last 26 amino acids and most preferably lacks the last 40 amino acids. The most preferred polynucleotides encoding Core that are suitable for use in the present invention are those that encode a truncated core containing the amino acids 1-171, 1-165, 1-151. Most preferably the polynucleotide encoding Core that is suitable for use in the present invention is that which encodes a truncated Core protein between amino acids 1-151. One or more consensus mutations as set forth in example 1 may be present.

The preferred NS4B polypeptide encoded by the polynucleotides of the present invention contain an N-terminal truncation to remove a region that is hypervariable between HCV isolates and genotypes. Preferably the NS4B polypeptide contains a deletion of between 30-100 amino acids from the N-terminus, more preferably between 40-80 amino acids, and most preferably a deletion of the first N-terminal 48 amino acids (in the context of the J4 L6 isolate this corresponds to a truncation at amino acid 1760, which is a loss of the first 48 amino acids of NS4B; equivalent truncations in other HCV isolates also form part of the present invention). Additionally, the NS4B sequence may be divided into two or more fragments and expressed in a polypeptide having the sequence of NS4B arranged in a different order to that found in the wild-type molecule.

The polynucleotides which are present in the vaccines of the present invention may comprise the natural nucleotide sequence as found in the HCV virus, however, it is preferred that the nucleotide sequence is codon optimised for expression in mammalian cells.

In addition to codon optimisation, it is preferred that the codon usage in the polynucleotides of the present invention encoding HCV Core, NS3, NS4B and NS5B is altered such that rare codons do not appear in concentrated clusters, and are on the contrary either relatively evenly spaced throughout the polynucleotide sequence, or are excluded from the codon optimised gene.

The DNA code has 4 letters (A, T, C and G) and uses these to spell three letter “codons” which represent the amino acids of the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons.

Where more than one codon is available to code for a given amino acid, it has been observed that the codon usage patterns of organisms are highly non-random. Different species show a different bias in their codon selection and, furthermore, utilisation of codons may be markedly different in a single species between genes which are expressed at high and low levels. This bias is different in viruses, plants, bacteria and mammalian cells, and some species show a stronger bias away from a random codon selection than others. For example, humans and other mammals are less strongly biased than certain bacteria or viruses. For these reasons, there is a significant probability that a mammalian gene expressed in E. coli or a viral gene expressed in mammalian cells will have an inappropriate distribution of codons for efficient expression. However, a gene with a codon usage pattern suitable for E. coli expression may also be efficiently expressed in humans. It is believed that the presence in a heterologous DNA sequence of clusters of codons which are rarely observed in the host in which expression is to occur, is predictive of low heterologous expression levels in that host.

There are several examples where changing codons from those which are rare in the host to those which are host-preferred (“codon optimisation”) has enhanced heterologous expression levels, for example the BPV (bovine papilloma virus) late genes L1 and L2 have been codon optimised for mammalian codon usage patterns and this has been shown to give increased expression levels over the wild-type HPV sequences in mammalian (Cos-1) cell culture (Zhou et. al. J. Virol 1999. 73, 4972-4982). In this work, every BPV codon which occurred more than twice as frequently in BPV than in mammals (ratio of usage >2), and most codons with a usage ratio of >1.5 were conservatively replaced by the preferentially used mammalian codon. In WO97/31115, WO97/48370 and WO98/34640 (Merck & Co., Inc.) codon optimisation of HIV genes or segments thereof has been shown to result in increased protein expression and improved immunogenicity when the codon optimised sequences are used as DNA vaccines in the host mammal for which the optimisation was tailored. In these documents, the sequences consist entirely of optimised codons (except where this would introduce an undesired restriction site, intron splice site etc.) because each viral codon is conservatively replaced with the optimal codon for the intended host.

The term “codon usage pattern” refers to the average frequencies for all codons in the nucleotide sequence, gene or class of genes under discussion (e.g. highly expressed mammalian genes). Codon usage patterns for mammals, including humans can be found in the literature (see e.g. Nakamura et. al. Nucleic Acids Research 1996, 24:214-215).

In the polynucleotides of the present invention, the codon usage pattern is preferably altered from that typical of HCV to more closely represent the codon bias of the target organism, e.g. E. coli or a mammal, especially a human. The “codon usage coefficient” or codon adaptation index (Sharp P M. Li W H. Nucleic Acids Research. 15(3):1281-95, 1987) is a measure of how closely the codon usage pattern of a given polynucleotide sequence resembles that of a target species. The codon frequencies for each of the 61 codons (expressed as the number of occurrences per 1000 codons of the selected class of genes) are normalised for each of the twenty natural amino acids, so that the value for the most frequently used codon for each amino acid is set to 1 and the frequencies for the less common codons are scaled proportionally to lie between zero and 1. Thus each of the 61 codons is assigned a value of 1 or lower for the highly expressed genes of the target species. This is referred to as the preference value (W). In order to calculate a codon usage coefficient for a specific polynucleotide, relative to the highly expressed genes of that species, the scaled value for each codon of the specific polynucleotide are noted and the geometric mean of all these values is taken (by dividing the sum of the natural logs of these values by the total number of codons and take the anti-log). The coefficient will have a value between zero and 1 and the higher the coefficient the more codons in the polynucleotide are frequently used codons. If a polynucleotide sequence has a codon usage coefficient of 1, all of the codons are “most frequent” codons for highly expressed genes of the target species.

The present invention provides polynucleotide sequences which encode HCV Core, NS3, NS4B or NS5B amino acid sequences, wherein the codon usage pattern of the polynucleotide sequence resembles that of highly expressed mammalian genes. Preferably the polynucleotide sequence is a DNA sequence. Desirably the codon usage pattern of the polynucleotide sequence resembles that of highly expressed human genes.

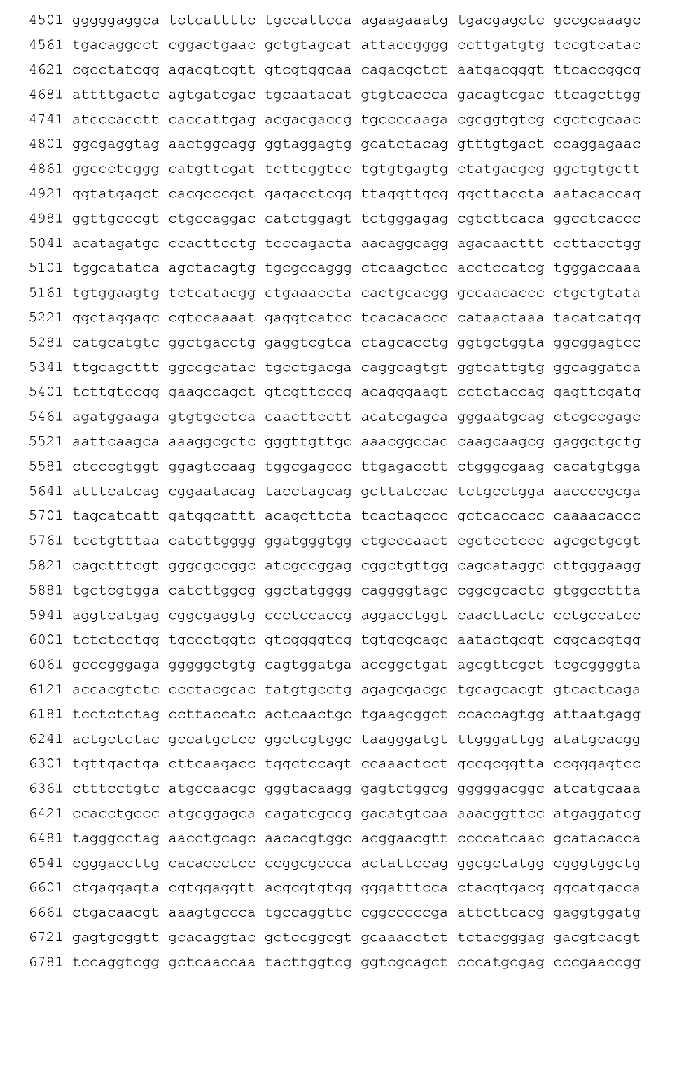

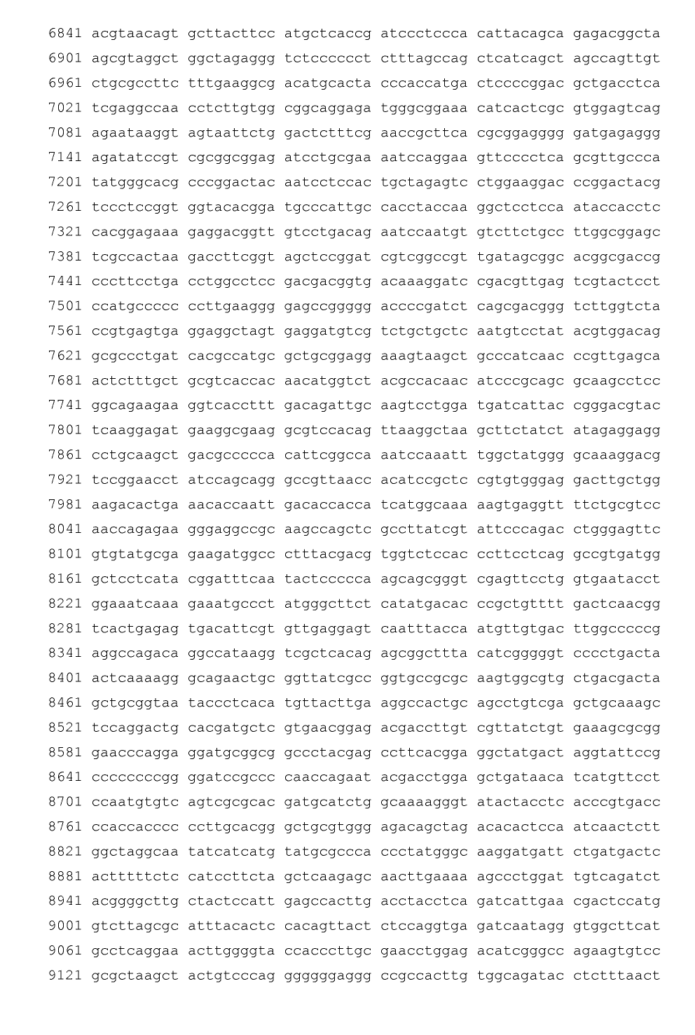

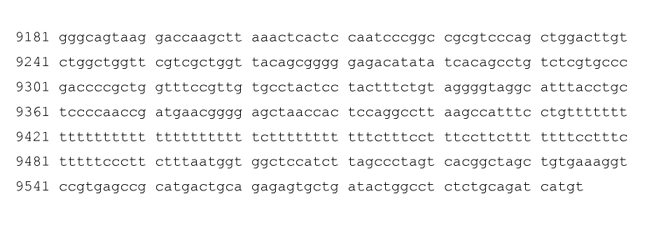

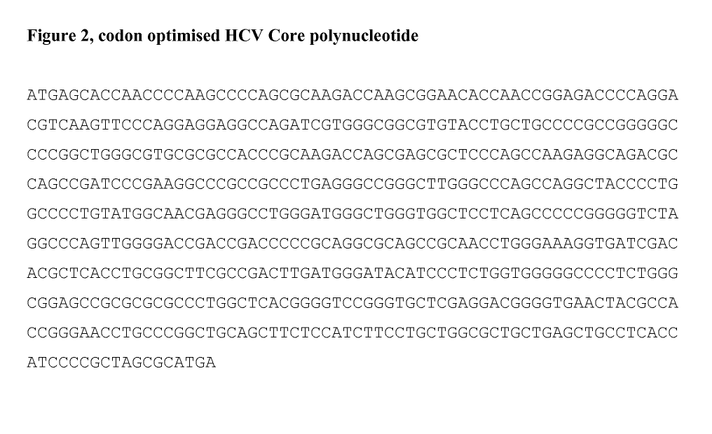

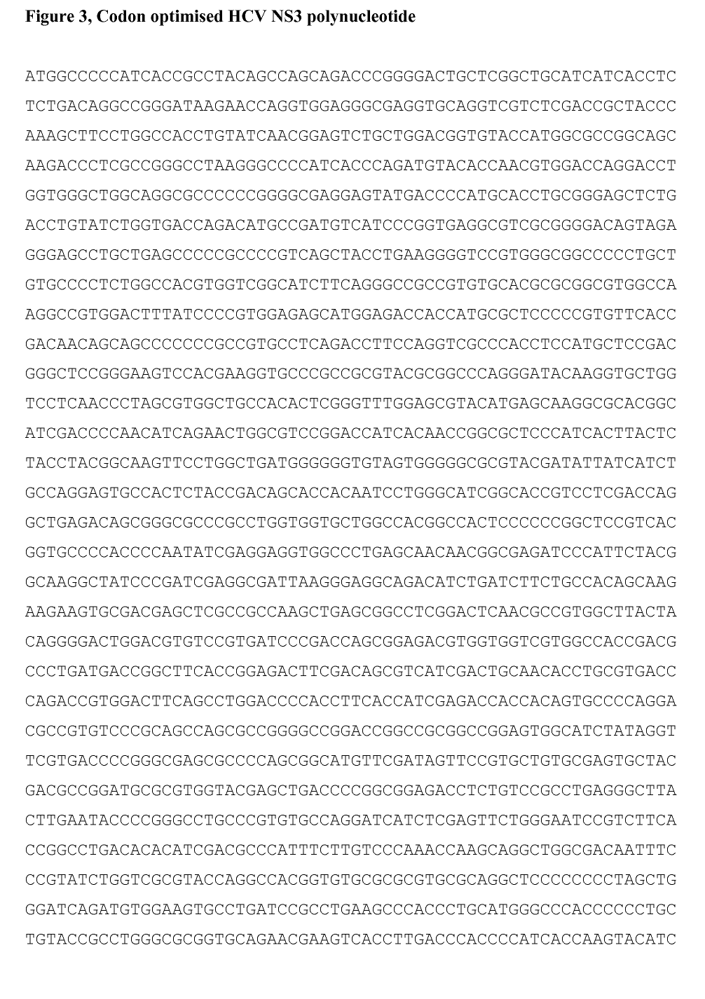

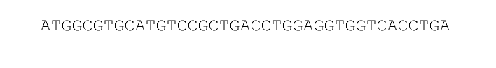

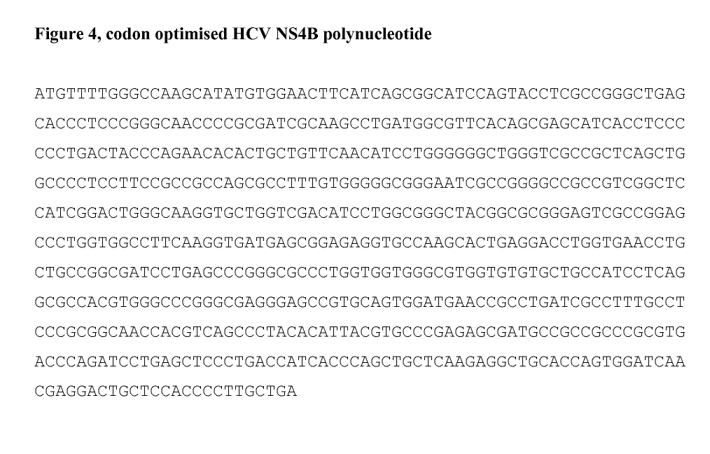

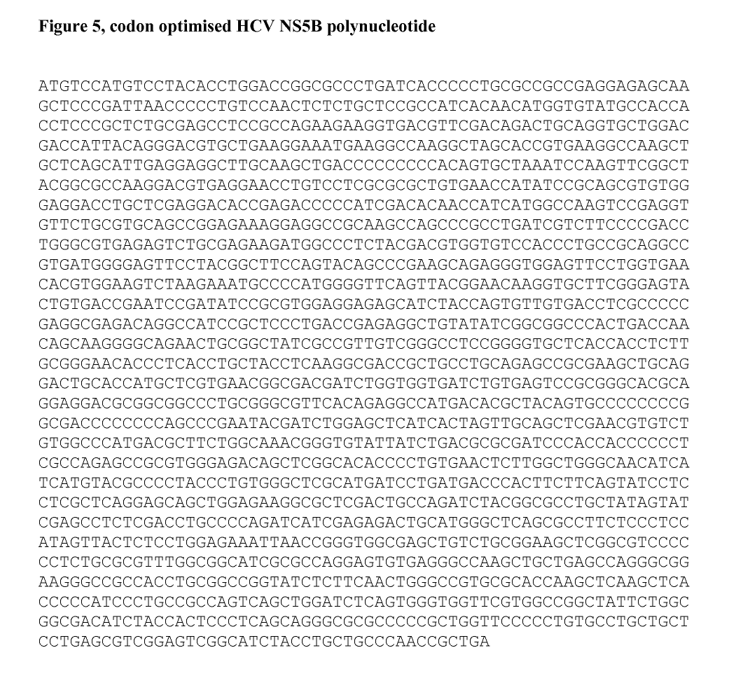

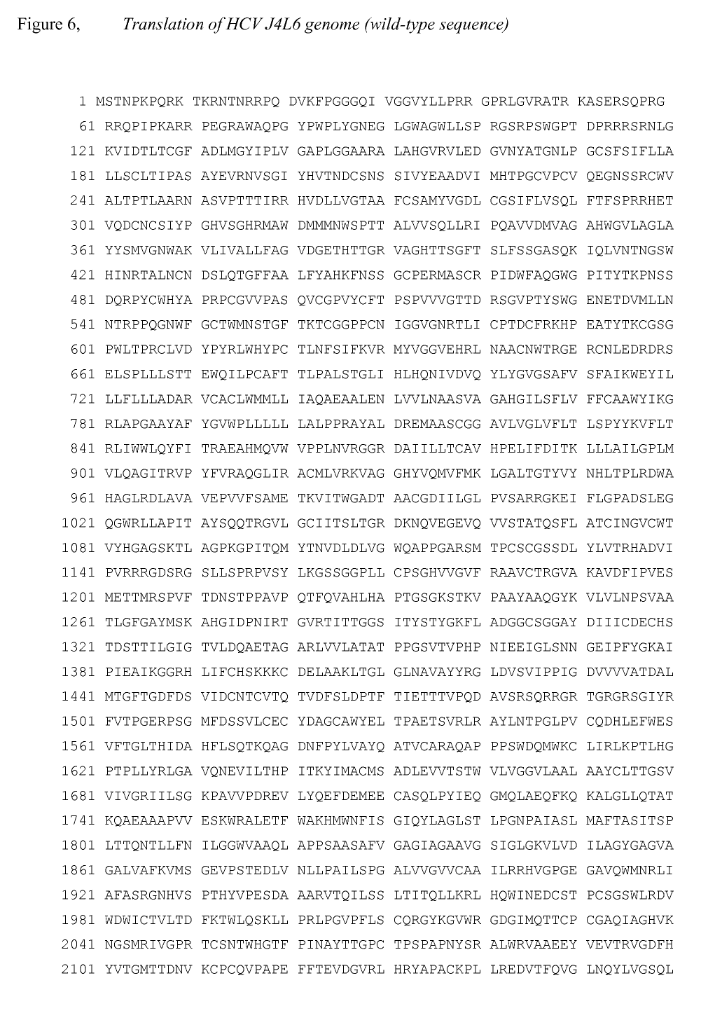

The codon optimised polynucleotide sequence encoding HCV core (1-191) is shown in FIG. 2. The codon optimised polynucleotide sequence encoding HCV NS3, comprising the S1165V and D1316Q polypeptide mutation, is shown in FIG. 3. The codon optimised polynucleotide sequence encoding HCV NS4B, comprising the N terminal 1-48 truncation of the polypeptide, is shown in FIG. 4. The codon optimised polynucleotide sequence encoding HCV NS5B, comprising the D2639G and D2644G polypeptide mutation, is shown in FIG. 5.

Accordingly, there is provided a synthetic gene comprising a plurality of codons together encoding HCV Core, NS3, NS4B or NS5B amino acid sequences, wherein the selection of the possible codons used for encoding the amino acid sequence has been changed to resemble the optimal mammalian codon usage such that the frequency of codon usage in the synthetic gene more closely resembles that of highly expressed mammalian genes than that of Hepatitis C virus genes. Preferably the codon usage pattern is substantially the same as that for highly expressed human genes. The “natural” HCV core, NS3, NS4B and NS5B sequences have been analysed for codon usage. The Codon usage coefficient for the HCV proteins are Core (0.487), NS3 (0.482), NS4B-0.481 and NS5B (0.459). A polynucleotide of the present invention will generally have a codon usage coefficient (as defined above) for highly expressed human genes of greater than 0.5, preferably greater than 0.6, most preferably greater than 0.7 but less than 1. Desirably the polynucleotide will also have a codon usage coefficient for highly expressed E. coli genes of greater than 0.5, preferably greater than 0.6, most preferably greater than 0.7.

In addition to Codon optimisation the synthetic genes are also mutated so as to exclude the appearance of clusters of rare codons. This can be achieved in one of two ways. The preferred way of achieving this is to exclude rare codons from the gene sequence. One method to define rare codons would be codons representing <20% of the codons used for a particular amino acid and preferably <10% of the codons used for a particular amino acid in highly expressed genes of the target organism. Alternatively rare codons may be defined as codons with a relative synonymous codon usage (RSCU) value of <0.3, or preferably <0.2 in highly expressed genes of the target organism. An RSCU value is the observed number of codons divided by the number expected if all codons for that amino acid were used equally frequently. An appropriate definition of a rare codon would be apparent to a person skilled in the art.

Alternatively the HCV core, NS3, NS4B and NS5B polynucleotides are optimised to prevent clustering of rare, non-optimal, codons being present in concentrated areas. The polynucleotides, therefore, are optimised such that individual rare codons, such as those with an RSCU of <0.4 (and more preferably of <0.3) are evenly spaced throughout the polynucleotides.

Expression levels of codon optimised mutated Core, NS3 and NS5B have been shown to be increased compared to wild type, as assessed by Western blot. The truncated codon optimised NS4B has been expressed as a fusion with NS5B, and the fusion expresses well.

The vaccines of the present invention may comprise a vector that directs individual expression of the HCV polypeptides, alternatively the HCV polypeptides may be expressed as one or more fusion proteins.

Preferred vaccines of the present invention comprise tetra-fusions either at the protein or polynucleotide level, including:

HCV combination 1: HCV 500

| | | | |
|---|---|---|---|
| Core | NS3 | NS4B | NS5B |

HCV combination 2: HCV 510

| | | | |
|---|---|---|---|
| NS3 | NS4B | NS5B | Core |

HCV combination 3: HCV 520

| | | | |
|---|---|---|---|
| NS4B | NS5B | Core | NS3 |

HCV combination 4: HCV 530

| | | | |
|---|---|---|---|
| NS5B | Core | NS3 | NS4B |

HCV combination 5: HCV 501

| | | | |
|---|---|---|---|
| Core (66-191)-(1-65) | NS3 | NS4B | NS5B |

HCV combination 6: HCV 502

| | | | |
|---|---|---|---|
| Core (105-191)-(1-104) | NS3 | NS4B | NS5B |

HCV combination 7:

| | | | |
|---|---|---|---|
| NS3 | NS4B | NS5B | Core 151 |

Other preferred fusions are analagous to HCV combinations 1, 2 and 3 but wherein the core protein is a truncated core protein, typically core 1-151. Other preferred vaccines of the present invention are given below and comprise polynucleotide double and triple fusions being present in different expression cassettes within the same plasmid, each cassette being under the independent control of a promoter unit (e.g. HCMV IE), (indicated by arrow). Such dual promoter constructs drive the expression of the four protein antigen as two separate proteins (as indicated below) in the same cell.

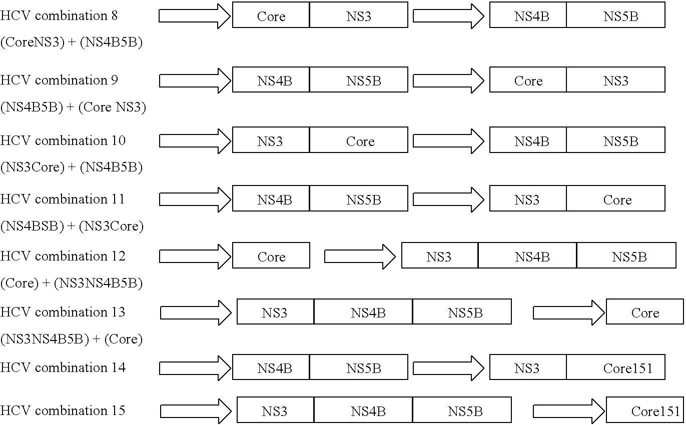

Preferred constructs are HCV combinations 7, 9, 11 or 12. Particularly preferred are 7 and 11.

In an alternative aspect of the present invention the polynucleotide vaccines optionally do not contain a polynucleotide encoding the core protein. For example, preferred polynucleotides of this aspect of the present invention include:

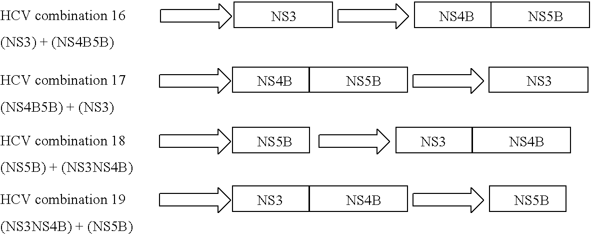

For HCV combinations 8-19 above, it is intended that the terminology used, eg. (CoreNS3)+(NS4B5B), is read to disclose a polynucleotide vector comprising two expression cassettes each independently controlled by a individual promoter, and in the case of this example, one expression cassette encoding a CoreNS3 double fusion protein and the other encoding a NS4B-NS5B double fusion protein. Each HCV combination 8-19 should be interpreted accordingly.

The above HCV combinations 1-19 disclose the relative orientations of the HCV proteins, polyprotein fusions, or polynucleotides. It is also specifically disclosed herein that all of the above HCV combinations 1-19 are also disclosed with each of the preferred mutations or truncations to remove the activity of the component proteins. For example, the preferred variants of the combinations 1-19 (unless otherwise indicated to the contrary) comprise the nucleotide sequences for Core (1-191 (all but divide sequence into two or more fragments to disable biological activity) or preferably Core being present in its truncated forms 1-151 or 1-165 or 1-171); NS3 1027-1657 (mutations to inactivate helicase (Aspartic acid 1316 to Glutamine) and protease (serine 1165 to valine) activity; NS5B 2420-3010 (mutation at Aspartic acid 2639 to Glycine and Aspartic acid 2644 to Glycine, Motif A) to inactivate polymerase activity); and NS4B 1712-1972 (optionally truncated to 1760-1972 remove N-terminal highly variable fragment).

The present invention provides the novel DNA vaccines and polypeptides as described above. Also provided by the present invention are analogues of the described polypeptides and DNA vaccines comprising them.

The term “analogue” refers to a polynucleotide which encodes the same amino acid sequence as another polynucleotide of the present invention but which, through the redundancy of the genetic code, has a different nucleotide sequence whilst maintaining the same codon usage pattern, for example having the same codon usage coefficient or a codon usage coefficient within 0.1, preferably within 0.05 of that of the other polynucleotide.

The HCV polynucleotide sequences may be derived from any of the various HCV genotypes, strains or isolates. HCV isolates can be classified into the following six major genotypes comprising one or more subtypes: HCV 1 (1a, 1b or 1c), HCV 2 (2a, 2b or 2c), HCV 3 (3a, 3b, 10a), HCV 4 (4a), HCV 5 (5a) and HCV 6 (6a, 6b, 7b, 8b, 9a and 11a); Simmonds, J. Gen. Virol., 2001, 693-712. In the context of the present invention each HCV protein may be derived from the polynucleotide sequence of the same HCV genotype or subtype, or alternatively any combination of HCV genotype or subtype, and HCV protein may be used. Preferably, the genes are derived from a type 1b genotype such as the infectious clone J4L6 (Accession No AF0542478—see FIG. 1).

Specific strains that have been sequenced include HCV-J (Kato et al., 1990, PNAS, USA, 87; 9724-9528) and BK (Takamizawa et al., 1991, J. Virol. 65:1105-1113).

The polynucleotides according to the invention have utility in the production by expression of the encoded proteins, which expression may take place in vitro, in vivo or ex vivo. The nucleotides may therefore be involved in recombinant protein synthesis, for example to increase yields, or indeed may find use as therapeutic agents in their own right, utilised in DNA vaccination techniques. Where the polynucleotides of the present invention are used in the production of the encoded proteins in vitro or ex vivo, cells, for example in cell culture, will be modified to include the polynucleotide to be expressed. Such cells include transient, or preferably stable mammalian cell lines. Particular examples of cells which may be modified by insertion of vectors encoding for a polyproteins according to the invention include mammalian HEK293T, CHO, HeLa, 293 and COS cells. Preferably the cell line selected will be one which is not only stable, but also allows for mature glycosylation and cell surface expression of a polyprotein. Expression may be achieved in transformed oocytes. A polypeptide may be expressed from a polynucleotide of the present invention, in cells of a transgenic non-human animal, preferably a mouse. A transgenic non-human animal expressing a polypeptide from a polynucleotide of the invention is included within the scope of the invention.

The present invention includes expression vectors that comprise the nucleotide sequences of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. Molecular Cloning: a Laboratory Manual. 2^{nd} Edition. CSH Laboratory Press. (1989).

Preferably, a polynucleotide of the invention, or for use in the invention in a vector, is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

An expression cassette is an assembly which is capable of directing the expression of the sequence or gene of interest. The expression cassette comprises control elements, such as a promoter which is operably linked to the gene of interest.

The vectors may be, for example, plasmids, artificial chromosomes (e.g. BAC, PAC, YAC), virus or phage vectors provided with a origin of replication, optionally a promoter for the expression of the polynucleotide and optionally a regulator of the promoter. The vectors may contain one or more selectable marker genes, for example an ampicillin or kanamycin resistance gene in the case of a bacterial plasmid or a resistance gene for a fungal vector. Vectors may be used in vitro, for example for the production of DNA or RNA or used to transfect or transform a host cell, for example, a mammalian host cell e.g. for the production of protein encoded by the vector. The vectors may also be adapted to be used in vivo, for example in a method of DNA vaccination or of gene therapy.

Promoters and other expression regulation signals may be selected to be compatible with the host cell for which expression is designed. For example, mammalian promoters include the metallothionein promoter, which can be induced in response to heavy metals such as cadmium, and the β-actin promoter. Viral promoters such as the SV40 large T antigen promoter, human cytomegalovirus (CMV) immediate early (IE) promoter, rous sarcoma virus LTR promoter, adenovirus promoter, or a HPV promoter, particularly the HPV upstream regulatory region (URR) may also be used. All these promoters are well described and readily available in the art.

Examples of suitable viral vectors include herpes simplex viral vectors, vaccinia or alpha-virus vectors and retroviruses, including lentiviruses, adenoviruses and adeno-associated viruses. Gene transfer techniques using these viruses are known to those skilled in the art. Retrovirus vectors for example may be used to stably integrate the polynucleotide of the invention into the host genome, although such recombination is not preferred. Replication-defective adenovirus vectors by contrast remain episomal and therefore allow transient expression. Vectors capable of driving expression in insect cells (for example baculovirus vectors), in human cells or in bacteria may be employed in order to produce quantities of the HCV protein encoded by the polynucleotides of the present invention, for example for use as subunit vaccines or in immunoassays.

In a further aspect, the present invention provides a pharmaceutical composition comprising a polynucleotide sequence as described herein. Preferably the composition comprises a DNA vector according to the second aspect of the present invention. In preferred embodiments the composition comprises a plurality of particles, preferably gold particles, coated with DNA comprising a vector encoding a polynucleotide sequence which encodes an HPV amino acid sequence, wherein the codon usage pattern of the polynucleotide sequence resembles that of highly expressed mammalian genes, particularly human genes. In alternative embodiments, the composition comprises a pharmaceutically acceptable excipient and a DNA vector according to the second aspect of the present invention. The composition may also include an adjuvant.

DNA vaccines may be delivered by interstitial administration of liquid vaccines into the muscle (WO90/11092) or by mechanisms other than intra-muscular injection. For example, delivery into the skin takes advantage of the fact that immune mechanisms are highly active in tissues that are barriers to infection such as skin and mucous membranes. Delivery into skin could be via injection, via jet injector (which forces a liquid into the skin, or underlying tissues including muscles, under pressure) or via particle bombardment, in which the DNA may be coated onto particles of sufficient density to penetrate the epithelium (U.S. Pat. No. 5,371,015). For example, the nucleotide sequences may be incorporated into a plasmid which is coated on to gold beads which are then administered under high pressure into the epidermis, such as, for example, as described in Haynes et al J. Biotechnology 44: 37-42 (1996). Projection of these particles into the skin results in direct transfection of both epidermal cells and epidermal Langerhan cells. Langerhan cells are antigen presenting cells (APC) which take up the DNA, express the encoded peptides, and process these for display on cell surface MHC proteins. Transfected Langerhan cells migrate to the lymph nodes where they present the displayed antigen fragments to lymphocytes, evoking an immune response. Very small amounts of DNA (less than 1 μg, often less than 0.5 g) are required to induce an immune response via particle mediated delivery into skin and this contrasts with the milligram quantities of DNA known to be required to generate immune responses subsequent to direct intramuscular injection.

Where the polynucleotides of the present invention find use as therapeutic agents, e.g. in DNA vaccination, the nucleic acid will be administered to the mammal e.g. human to be vaccinated. The nucleic acid, such as RNA or DNA, preferably DNA, is provided in the form of a vector, such as those described above, which may be expressed in the cells of the mammal. The polynucleotides may be administered by any available technique. For example, the nucleic acid may be introduced by needle injection, preferably intradermally, subcutaneously or intramuscularly. Alternatively, the nucleic acid may be delivered directly into the skin using a nucleic acid delivery device such as particle-mediated DNA delivery (PMDD). In this method, inert particles (such as gold beads) are coated with a nucleic acid, and are accelerated at speeds sufficient to enable them to penetrate a surface of a recipient (e.g. skin), for example by means of discharge under high pressure from a projecting device. (Particles coated with a nucleic acid molecule of the present invention are within the scope of the present invention, as are delivery devices loaded with such particles). The composition desirably comprises gold particles having an average diameter of 0.5-5 μm, preferably about 2 μm. In preferred embodiments, the coated gold beads are loaded into tubing to serve as cartridges such that each cartridge contains 0.1-1 mg, preferably 0.5 mg gold coated with 0.1-5 μg, preferably about 0.5 μg DNA/cartridge.

According to another aspect of the invention there is provided a host cell comprising a polynucleotide sequence as described herein. The host cell may be bacterial, e.g. E. coli, mammalian, e.g. human, or may be an insect cell. Mammalian cells comprising a vector according to the present invention may be cultured cells transfected in vitro or may be transfected in vivo by administration of the vector to the mammal.

In a further aspect, the present invention provides a method of making a pharmaceutical composition as described above, including the step of altering the codon usage pattern of a wild-type HCV nucleotide sequence, or creating a polynucleotide sequence synthetically, to produce a sequence having a codon usage pattern resembling that of highly expressed mammalian genes and encoding a wild-type HCV amino acid sequence or a mutated HCV amino acid sequence comprising the wild-type sequence with amino acid changes sufficient to inactivate one or more of the natural functions of the polypeptide.

Also provided are the use of a polynucleotide or vaccine as described herein, in the treatment or prophylaxis of an HCV infection.

Suitable techniques for introducing the naked polynucleotide or vector into a patient include topical application with an appropriate vehicle. The nucleic acid may be administered topically to the skin, or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by use of facilitating agents such as bupivacaine, either separately or included in the DNA formulation. Other methods of administering the nucleic acid directly to a recipient include ultrasound, electrical stimulation, electroporation and microseeding which is described in U.S. Pat. No. 5,697,901.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in an amount in the range of 1 pg to 1 mg, preferably 1 pg to 10 μg nucleic acid for particle mediated gene delivery and 10 μg to 1 mg for other routes.

A nucleic acid sequence of the present invention may also be administered by means of specialised delivery vectors useful in gene therapy. Gene therapy approaches are discussed for example by Verme et al, Nature 1997, 389:239-242. Both viral and non-viral vector systems can be used. Viral based systems include retroviral, lentiviral, adenoviral, adeno-associated viral, herpes viral, Canarypox and vaccinia-viral based systems. Preferred adenoriral vectors are those derived from non-human primates. In particular Pan 9 (C68) as described in U.S. Pat. No. 6,083,716, Pan5, 6 or 7 as described in WO03/046124.

Non-viral based systems include direct administration of nucleic acids, microsphere encapsulation technology (poly(lactide-co-glycolide) and, liposome-based systems. Viral and non-viral delivery systems may be combined where it is desirable to provide booster injections after an initial vaccination, for example an initial “prime” DNA vaccination using a non-viral vector such as a plasmid followed by one or more “boost” vaccinations using a viral vector or non-viral based system. Prime boost protocols may also take advantage of priming with protein in adjuvant and boosting with DNA or a viral vector encoding the polynucleotide of the invention. Alternatively the protein based vaccine may be used as a booster. It is preferred that the protein vaccine will contain all the antigens that the DNA/viral vectored vaccine contain. The proteins however, maybe presented individually or as a polyprotein.

A nucleic acid sequence of the present invention may also be administered by means of transformed cells. Such cells include cells harvested from a subject. The naked polynucleotide or vector of the present invention can be introduced into such cells in vitro and the transformed cells can later be returned to the subject. The polynucleotide of the invention may integrate into nucleic acid already present in a cell by homologous recombination events. A transformed cell may, if desired, be grown up in vitro and one or more of the resultant cells may be used in the present invention. Cells can be provided at an appropriate site in a patient by known surgical or microsurgical techniques (e.g. grafting, micro-injection, etc.)

Suitable cells include antigen-presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumour, e.g. anti-cervical carcinoma effects per se and/or to be immunologically compatible with the receiver (i.e., matched HLA haplotype). APCs may generally be isolated from any of a variety of biological fluids and organs, including tumour and peri-tumoural tissues, and may be autologous, allogeneic, syngeneic or xenogeneic cells.

Certain preferred embodiments of the present invention use dendritic cells or progenitors thereof as antigen-presenting cells, either for transformation in vitro and return to the patient or as the in vivo target of nucleotides delivered in the vaccine, for example by particle mediated DNA delivery. Dendritic cells are highly potent APCs (Banchereau and Steinman, Nature 392:245-251, 1998) and have been shown to be effective as a physiological adjuvant for eliciting prophylactic or therapeutic antitumour immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naïve T cell responses. Dendritic cells may, of course, be engineered to express specific cell-surface receptors or ligands that are not commonly found on dendritic cells in vivo or ex vivo, for example the antigen(s) encoded in the constructs of the invention, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumour-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNF to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNF, CD40 ligand, lipopolysaccharide LPS, flt3 ligand (a cytokine important in the generation of professional antigen presenting cells, particularly dendritic cells) and/or other compound(s) that induce differentiation, maturation and proliferation of dendritic cells.

APCs may generally be transfected with a polynucleotide encoding an antigenic HCV amino acid sequence, such as a codon-optimised polynucleotide as envisaged in the present invention. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the particle mediated approach described by Mahvi et al., Immunology and cell Biology 75:456-460, 1997.

The Vaccines and pharmaceutical compositions of the invention may be used in conjunction with antiviral agents such as α-interferon, preferably pegalated α-interferon, and a ribovarin. Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use. Vaccines comprising nucleotide sequences intended for administration via particle mediated delivery may be presented as cartridges suitable for use with a compressed gas delivery instrument, in which case the cartridges may consist of hollow tubes the inner surface of which is coated with particles bearing the vaccine nucleotide sequence, optionally in the presence of other pharmaceutically acceptable ingredients.

The pharmaceutical compositions of the present invention may include adjuvant compounds, or other substances which may serve to modulate or increase the immune response induced by the protein which is encoded by the DNA. These may be encoded by the DNA, either separately from or as a fusion with the antigen, or may be included as non-DNA elements of the formulation. Examples of adjuvant-type substances which may be included in the formulations of the present invention include ubiquitin, lysosomal associated membrane protein (LAMP), hepatitis B virus core antigen, flt3-ligand and other cytokines such as IFN-γ and GMCSF.

Other suitable adjuvants are commercially available such as, for example, Freund's Incomplete Adjuvant and Complete Adjuvant (Difco Laboratories, Detroit, Mich.); Imiquimod (3M, St. Paul, Minn.); Resimiquimod (3M, St. Paul, Minn.); Merck Adjuvant 65 (Merck and Company, Inc., Rahway, N.J.); aluminium salts such as aluminium hydroxide gel (alum) or aluminium phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

In the formulations of the invention it is preferred that the adjuvant composition induces an immune response predominantly of the Th1 type. Thus the adjuvant may serve to modulate the immune response generated in response to the DNA-encoded antigens from a predominantly Th2 to a predominantly Th1 type response. High levels of Th1-type cytokines (e.g., IFN-, TNF, IL-2 and IL-12) tend to favour the induction of cell mediated immune responses to an administered antigen. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Accordingly, suitable adjuvants for use in eliciting a predominantly Th1-type response include, for example, a combination of monophosphoryl lipid A, preferably 3-de-O-acylated monophosphoryl lipid A (3D-MPL) together with an aluminium salt. Other known adjuvants which preferentially induce a TH1 type immune response include CpG containing oligonucleotides. The oligonucleotides are characterised in that the CpG dinucleotide is unmethylated. Such oligonucleotides are well known and are described in, for example WO96/02555. Immunostimulatory DNA sequences are also described, for example, by Sato et al., Science 273:352, 1996. CpG-containing oligonucleotides may be encoded separately from the papilloma antigen(s) in the same or a different polynucleotide construct, or may be immediately adjacent thereto, e.g. as a fusion therewith. Alternatively the CpG-containing oligonucleotides may be administered separately i.e. not as part of the composition which includes the encoded antigen. CpG oligonucleotides may be used alone or in combination with other adjuvants. For example, an enhanced system involves the combination of a CpG-containing oligonucleotide and a saponin derivative particularly the combination of CpG and QS21 as disclosed in WO 00/09159 and WO 00/62800. Preferably the formulation additionally comprises an oil in water emulsion and/or tocopherol.

Another preferred adjuvant is a saponin, preferably QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

Other preferred adjuvants include Montanide ISA 720 (Seppic, France), SAF (Chiron, Calif., United States), ISCOMS (CSL), MF-59 (Chiron), Detox (Ribi, Hamilton, Mont.), RC-529 (Corixa, Hamilton, Mont.) and other aminoalkyl glucosaminide 4-phosphates (AGPs).

Where the vaccine includes an adjuvant, the vaccine formulation may be administered in two parts. For example, the part of the formulation containing the nucleotide construct which encodes the antigen may be administered first, e.g. by subcutaneous or intramuscular injection, or by intradermal particle-mediated delivery, then the part of the formulation containing the adjuvant may be administered subsequently, either immediately or after a suitable time period which will be apparent to the physician skilled in the vaccines arts. Under these circumstances the adjuvant may be administered by the same route as the antigenic formulation or by an alternate route. In other embodiments the adjuvant part of the formulation will be administered before the antigenic part. In one embodiment, the adjuvant is administered as a topical formulation, applied to the skin at the site of particle mediated delivery of the nucleotide sequences which encode the antigen(s), either before or after the particle mediated delivery thereof.

Preferably the DNA vaccines of the present invention stimulate an effective immune response, typically CD4+ and CD8+ immunity against the HCV antigens. Preferably against a broad range of epitopes. It is preferred in a therapeutic setting that liver fibrosis and/or inflammation be reduced following vaccination.

As used herein, the term comprising is intended to be used in its non-limiting sense such that the presence of other elements is not excluded. However, it is also intended that the word “comprising” could also be understood in its exclusive sense, being commensurate with “consisting” or “consisting of”. The present invention is illustrated, but not limited to, the following examples.

### EXAMPLE 1

### Mutations Introduced into Antigen Panel

### 1). Consensus Mutations

A comparison of the full genome sequences of all known HCV isolates was carried out. Certain positions within the J4L6 polyprotein were identified as unusual/deviating from the majority of other HCV isolates. With particular importance were those positions found to deviate from a more consensus residue across related 1b-group isolates, extending across groups 1a, 2, 3, and others, where one or two alternative amino acid residues otherwise dominated in the equivalent position. None of the chosen consensus mutations interferes with a known CD4 or CD8 epitope. Two changes within NS3 actually restore an immunodominant HLA-B35-restricted CD8 epitope [Isoleucine (1) 1365 to Valine (V) and Glycine (G) 1366 to Alanine (A)].

The first 51 amino acids of NS4B have been removed due to unuseful variability.

### Core

Alanine (A) 52 to Threonine (T)

### NS3

Valine (V) 1040 to Leucine (L)

Leucine (L) 1106 to Glutamine (Q)

Serine (S) 1124 to Threonine (T)

Valine (V) 1179 to Isoleucine (I)

Threonine (T) 1215 to Serine (S)

Glycine (G) 1289 to Alanine (A)

Serine (S) 1290 to Proline (P)

Isoleucine (1) 1365 to Valine (V)

Glycine (G) 1366 to Alanine (A)

Threonine (T) 1408 to Serine (S)

Proline (P) 1428 to Threonine (T)

Isoleucine (1) 1429 to Serine (S)

Isoleucine (1) 1636 to Threonine (T)

### NS4B

Start ORF at Phenylalanine (F) 1760

### NS5B

### Isoleucine (I) 2824 to Valine (V)

### Threonine (T) 2892 to Serine (S)

### Threonine (T) 2918 to Valine (V)

N.B. Numbering is according to position in polyprotein for J4L6 isolate.

### EXAMPLE 2

### Construction of Plasmid DNA Vaccines

Polynucleotide sequences encoding HCV Core, NS3, truncated NS4B, and NS5B, were codon optimised for mammalian codon usage using SynGene 2e software. The codon usage coefficient was improved to greater than 0.7 for each polynucleotide. The sense and anti-sense strands of each new polynucleotide sequence, incorporating codon optimisation, enzymatic knockout mutations, and consensus mutations, were divided into regions of 40-60 nucleotides, with a 20 nucleotide overlap. These regions were synthesised commercially and the polynucleotide generated by an oligo assembly PCR method.

The outer forward and reverse PCR primers for each polynucleotide, illustrating unique restriction endonuclease sites used for cloning, are outlined below:

```
HCV Core
Forward primer
5′-GAATTCGCGGCCGCCATGAGCACCAACCCCAAGCCCCAGCGCAAGACCAAGCGGAACACC-3′
                  NotI translation
                                start codon
Reverse primer
5′-GAATTCGGATCCTCATGCGCTAGCGGGGATGGTGAGGCAGCTCAGCAGCGCCAGCAGGA-3′
                  BamHI Stop
                              codon
HCV NS3
Forward primer
5′-GAATTCGCGGCCGCCATGGCCCCCATCACCGCCTACAGCCAGCAGACCCGGGGAC-3′
                  NotI translation
                                start codon
Reverse primer
5′-GAATTCGGATCCTCAGGTGACCACCTCCAGGTCAGCGGACATGCACGCCATGATG-3′
                  BamHI Stop
                              codon
HCV NS4B
Forward primer
5′-GAATTCGCGGCCGCCATGTTTTGGGCCAAGCATATGTGGAACTTCA-3′
                  NotI translation
                                start codon
Reverse primer
5′-GAATTCGGATCCTCAGCAAGGGGTGGAGCAGTCCTCGTTGATCCAC-3′
                  BamHI Stop
                              codon
HCV NS5B
Forward primer
5′-GAATTCGCGGCCGCCATGTCCATGTCCTACACCTGGACCGGCGCCCTGA-3′
                  NotI translation
                                start codon
Reverse primer
5′-GAATTCGGATCCTCAGCGGTTGGGCAGCAGGTAGATGCCGACTCCGACG-3′
                  BamHI Stop
                              codon
```

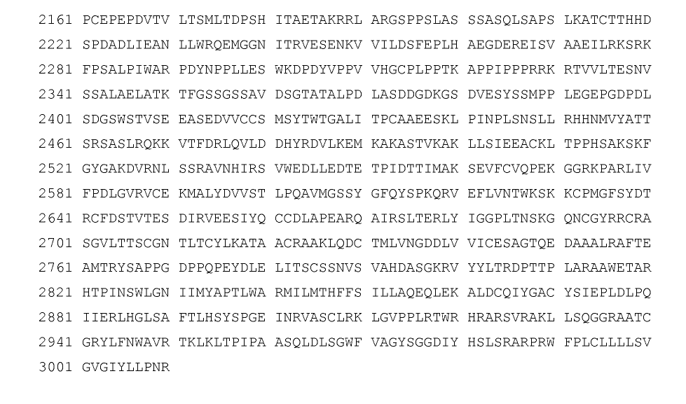

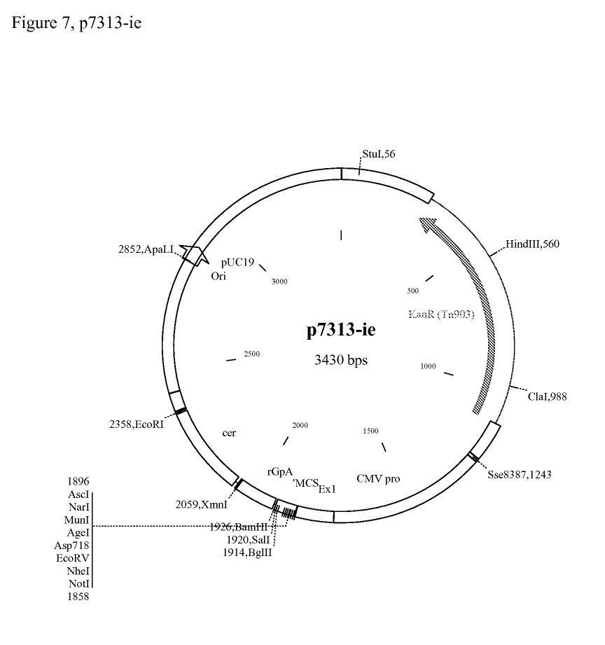

All polynucleotides, encoding single antigens, were cloned into mammalian expression vector p7313ie via Not I and BamHI unique cloning sites (see FIG. 7).

The polyproteins that were encoded were as follows (including mutations and codon optimisations):

HCV Core translation:
MSTNPKPQRKTKRNTNRRPQDVKFPGGGQIVGGVYLLPRRGPRLGVRATR
KTSERSQPRGRRQPIPKARRPEGRAWAQPGYPWPLYGNEGLGWAGWLLSP
RGSRPSWGPTDPRRRSRNLGKVIDTLTCGFADLMGYIPLVGAPLGGAARA
LAHGVRVLEDGVNYATGNLPGCSFSIFLLALLSCLTIPASA
HCV NS3 translation:
MAPITAYSQQTRGLLGCIITSLTGRDKNQVEGEVQVVSTATQSFLATCIN
GVCWTVYHGAGSKTLAGPKGPITQMYTNVDQDLVGWQAPPGARSMTPCTC
GSSDLYLVTRHADVIPVRRRGDSRGSLLSPRPVSYLKGSVGGPLLCPSGH
VVGIFRAAVCTRGVAKAVDFIPVESMETTMRSPVFTDNSSPPAVPQTFQV
AHLHAPTGSGKSTKVPAAYAAQGYKVLVLNPSVAATLGFGAYMSKAHGID
PNIRTGVRTITTGAPITYSTYGKFLADGGCSGGAYDIIICQECHSTDSTT
ILGIGTVLDQAETAGARLVVLATATPPGSVTVPHPNIEEVALSNNGEIPF
YGKAIPIEAIKGGRHLIFCHSKKKCDELAAKLSGLGLNAVAYYRGLDVSV
IPTSGDVVVVATDALMTGFTGDFDSVIDCNTCVTQTVDFSLDPTFTIETT
TVPQDAVSRSQRRGRTGRGRSGIYRFVTPGERPSGMFDSSVLCECYDAGC
AWYELTPAETSVRLRAYLNTPGLPVCQDHLEFWESVFTGLTHIDAHFLSQ
TKQAGDNFPYLVAYQATVCARAQAPPPSWDQMWKCLIRLKPTLHGPTPLL
YRLGAVQNEVTLTHPITKYIMACMSADLEVVT
HCV NS4B translation:
MFWAKHMWNFISGIQYLAGLSTLPGNPAIASLMAFTASITSPLTTQNTLL
FNILGGWVAAQLAPPSAASAFVGAGIAGAAVGSIGLGKVLVDILAGYGAG
VAGALVAFKVMSGEVPSTEDLVNLLPAILSPGALVVGVVCAAILRRHVGP
GEGAVQWMNRLIAFASRGNHVSPTHYVPESDAAARVTQILSSLTITQLLK
RLHQWINEDCSTPC
HCV NS5B translation:
MSMSYTWTGALITPCAAEESKLPINPLSNSLLRHHNMVYATTSRSASLRQ
KKVTFDRLQVLDDHYRDVLKEMKAKASTVKAKLLSIEEACKLTPPHSAKS
KFGYGAKDVRNLSSRAVNHIRSVWEDLLEDTETPIDTTIMAKSEVFCVQP
EKGGRKPARLIVFPDLGVRVCEKMALYDVVSTLPQAVMGSSYGFQYSPKQ
RVEFLVNTWKSKKCPMGFSYGTRCFGSTVTESDIRVEESIYQCCDLAPEA
RQAIRSLTERLYIGGPLTNSKGQNCGYRRCRASGVLTTSCGNTLTCYLKA
TAACRAAKLQDCTMLVNGDDLVVICESAGTQEDAAALRAFTEAMTRYSAP
PGDPPQPEYDLELITSCSSNVSVAHDASGKRVYYLTRDPTTPLARAAWET
ARHTPVNSWLGNIIMYAPTLWARMILMTHFFSILLAQEQLEKALDCQIYG
ACYSIEPLDLPQIIERLHGLSAFSLHSYSPGEINRVASCLRKLGVPPLRV
WRHRARSVRAKLLSQGGRAATCGRYLFNWAVRTKLKLTPIPAASQLDLSG
WFVAGYSGGDIYHSLSRARPRWFPLCLLLLSVGVGIYLLPNR

### EXAMPLE 3

### Immune Response Assays

C57BL or BALB/c mice were immunised with either WT or codon optimised+mutated versions of the four HCV antigens expressed individually in the p7313 vector. Mice were immunised by PMID with a standard dose of 1.0 μg/cartridge and boosted and day 21 (boost 1), and again at day 49 (boost 2). Spleen cells were harvested from individual mice and restimulated in ELISPOT with different HCV antigen preparations. Both IL2 and IFNγ responses were measured. The reagents used to measure immune responses were purified HCV core, NS3, NS4 and NS5B (genotype 1b) proteins from Mikrogen, Vacinnia-Core and Vaccinia NS3-5 (genotype 1b in house).

### HCV Core

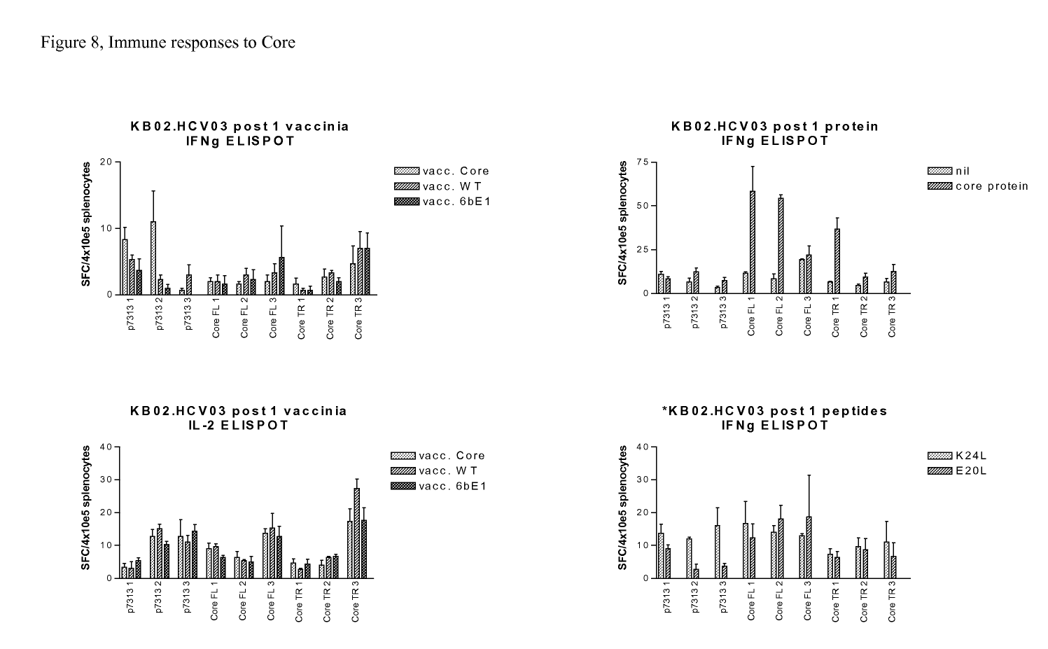

C57BL Mice immunised with WT full length (FL-1-191) or truncated (TR 1-115) core were restimulated with HCV core protein and good responses were observed with purified core protein (FIG. 8)

### HCV NS3

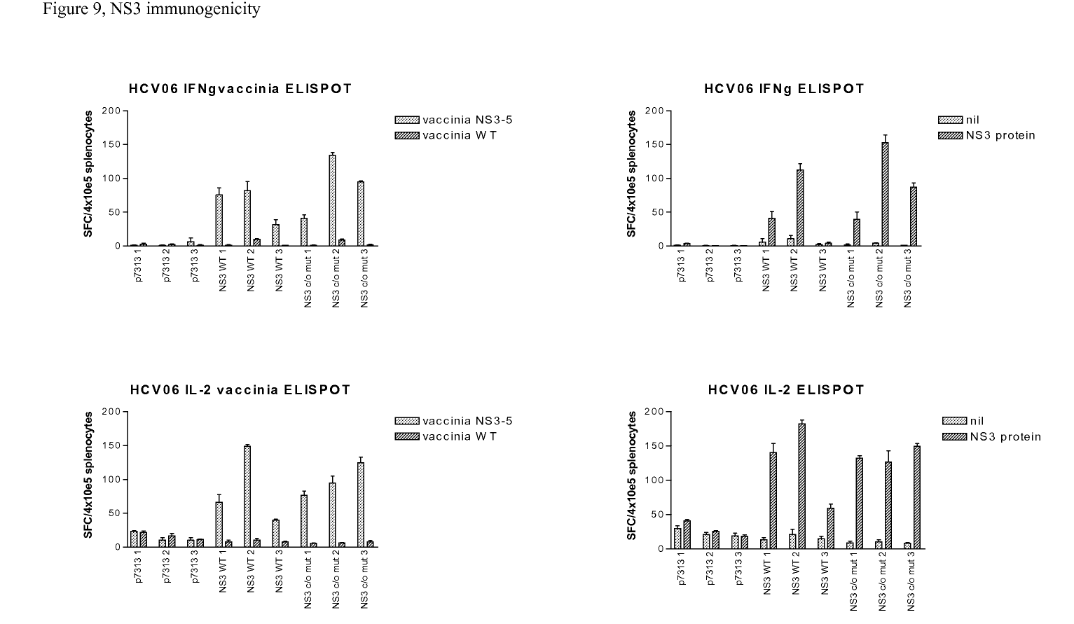

Mice were immunised with p7313 WT and codon optimised NS3 using PMID. Good responses to NS3 following immunisation and a single boost were demonstrated in C57B1 mice using both NS3 protein and Vaccinia 3-5 to read out the response by ELISPOT. Both IL2 and IFNγ responses were detected. No significant differences between wild type and codon optimised (co+m) versions of the constructs were observed in this experiment (FIG. 9). However differences in in vitro expression following transient transfection were observed between wild type and codon optimised constructs. Experiments to compare constructs at lower DNA dose or in the primary response may reveal differences in the potency of the plasmids.

### HCV NS4B

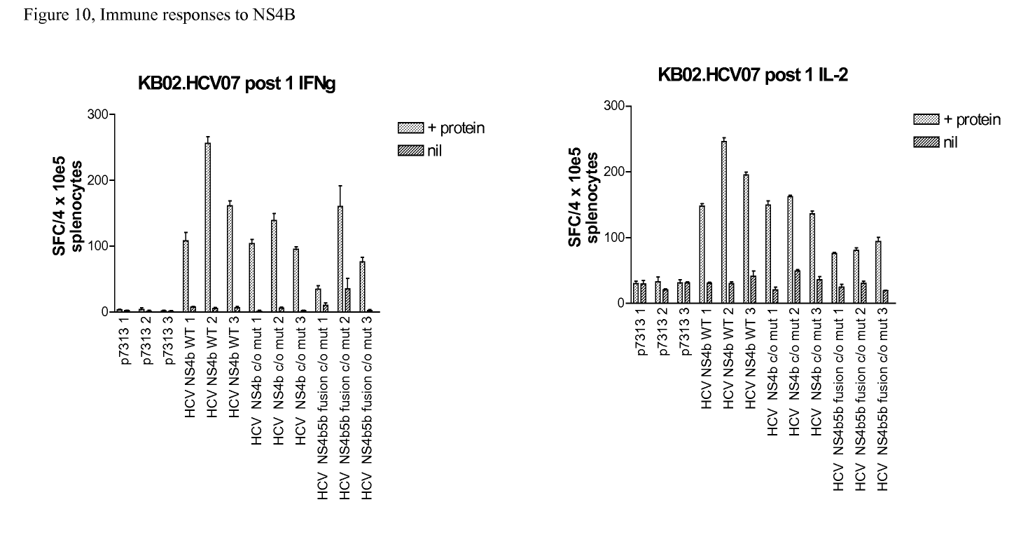

Responses to full length WT p7313 NS4B were observed following PMID immunisation of BALB/c mice. Both IL2 and IFNγ ELISPOT responses were observed following in vitro restimulation with either NS4B protein and Vaccinia 3-5 (FIG. 10).

The NS4B protein was truncated at the N-terminus to remove a highly variable region, however expression of this protein could not be detected following in vitro tranfection studies because the available anti-sera had been raised against the N-terminal region. In order to confirm expression of this region it was fused with the NS5B protein. Recent experiments have confirmed that immune responses can be detected against the truncated NS4B protein, either alone or as a fusion with NS5B, using the NS4B protein and NS3-5 vaccinia. Good responses were observed to WT and codon optimised NS4B.

### HCV NS5B

The immune response to NS5B following PMID was investigated following immunisation with WT and codon optimised (co+M) sequences. Good responses to NS5B following immunisation and a single boost were demonstrated in C57BL mice using both NS3 protein and vaccinia 3-5 to read out the response by ELISPOT. As with NS3 no differences in the immune response were observed between WT and co+m versions of the constructs in this experiment (FIG. 11).

### EXAMPLE 4

### Expression of HCV Polyproteins

The four selected HCV antigens Core, NS3, NS4B and NS5B were formatted in p7313ie to express as a single fusion polyprotein. The antigens were expressed in a different order in the different constructs as shown below. (The construct panel encoding the expression of single polyproteins was designed so the amino-terminal position was taken by each of the four antigens in turn, to monitor whether the level of expression was significantly improved or reduced more by the presence of one antigen than another in this important position.) In addition two constucts were generated in which the Core protein was re-arranged into in to 2 fragments ie Core 66-191>1-65 and 105-191>1-104.

HCV 500

| | | | |
|---|---|---|---|
| Core | NS3 | NS4B | NS5B |

HCV 510

| | | | |
|---|---|---|---|
| NS3 | NS4B | NS5B | Core |

HCV 520

| | | | |
|---|---|---|---|
| NS4B | NS5B | Core | NS3 |

HCV 530

| | | | |
|---|---|---|---|
| NS5B | Core | NS3 | NS4B |

HCV 501

| | | | |
|---|---|---|---|
| Core (66-191)-(1-65) | NS3 | NS4B | NS5B |

HCV 502

| | | | |
|---|---|---|---|
| Core (105-191)-(1-104) | NS3 | NS4B | NS5B |

A standardised amount of DNA was transfected into HEK 293T cells using Lipofectamine 2000 transfection reagent (Invitrogen/Life Technologies), following the standard manufacturers protocol. Cells were harvested 24 hours post-transfection, and polyacrylamide gel electrophoresis carried out using NuPAGE 4-12% Bis-Tris pre-formed gels with either MOPS or MES ready-made buffers (Invitrogen/Life Technologies). The separated proteins were blotted onto PVDF membrane and protein expression monitored using rabbit antiserum raised against NS5B whole protein. The secondary probe was an anti-rabbit immunoglobulin antiserum conjugated to horseradish peroxidase (hrp), followed by chemi-luminescent detection using ECL reagents (Amersham Biosciences).

The results of this expression study are shown in FIG. 12. The results show that all the polyproteins are expressed to similar extent although at lower levels than that seen to single antigen expressing NS5B. The slightly lower molecular weight of HCV500 is due to cleavage of HCV core from the N-terminal position. HCV502 was not detected in this experiment due to a cloning error. In a repeat experiment with another clone the level of expression of HCV502 was similar to the other polyproteins.

### EXAMPLE 5

### Detection of Immune response to HCV Polyproteins

C57BL mice were immunised by PMID with DNA (1 μg) encoding each of the polyproteins, followed by boosting 3 weeks later as described in example 4. Immune responses were monitored 7 days post boost using ELISPOT or intracellular cytokine production to the HCV antigens.

### ELISPOT Assays for T Cell Responses to HCV Gene Products

### Preparation of Splenocytes

Spleens were obtained from immunised animals at 7 days post boost. Spleens were processed by grinding between glass slides to produce a cell suspension. Red blood cells were lysed by ammonium chloride treatment and debris was removed to leave a fine suspension of splenocytes. Cells were resuspended at a concentration of 4×10⁶/ml in RPMI complete media for use in ELISPOT assays where mice had received only a primary immunisation and 2×10⁶/ml where mice had been boosted.

### ELISPOT Assay

Plates were coated with 15 μg/ml (in PBS) rat anti mouse IFNγ or rat anti mouse IL-2 (Pharmingen). Plates were coated overnight at +4° C. Before use the plates were washed three times with PBS. Splenocytes were added to the plates at 4×105 cells/well. Recombinant HCV antigens were obtained from Mikrogen and used at 1 ug/ml. Peptide was used in assays at a final concentration of 1-10 uM to measure CD4 or CD8 responses. These peptides were obtained from Genemed Synthesis. Total volume in each well was 200 μl. Plates containing antigen stimulated cells were incubated for 16 hours in a humidified 37° C. incubator. In some experiments cells infected with recombinant Vaccinia expressing NS3-5 or Vaccinia Wild type were used as antigens in ELISPOT assay.

### Development of ELISPOT Assay Plates.

Cells were removed from the plates by washing once with water (with 1 minute soak to ensure lysis of cells) and three times with PBS. Biotin conjugated rat anti mouse IFN-γ or IL-2 (Phamingen) was added at 1 μg/ml in PBS. Plates were incubated with shaking for 2 hours at room temperature. Plates were then washed three times with PBS before addition of Streptavidin alkaline phosphatase (Caltag) at 1/1000 dilution. Following three washes in PBS spots were revealed by incubation with BCICP substrate (Biorad) for 15-45 mins. Substrate was washed off using water and plates were allowed to dry. Spots were enumerated using an image analysis system.

Flow Cytometry to Detect IFNγ and IL2 Production from T Cells in Response to Peptide Stimulation.

Approximately 3×106 splenocytes were aliquoted per test tube, and spun to pellet. The supernatant was removed and samples vortexed to break up the pellet. 0.5 μg of anti-CD28+0.5 μg of anti-CD49d (Pharmingen) were added to each tube, and left to incubate at room temperature for 10 minutes. 1 ml of medium was added to appropriate tubes, which contained either medium alone, or medium with HCV antigens. Samples were then incubated for an hour at 37° C. in a heated water bath. 10 ug/ml Brefeldin A was added to each tube and the incubation at 37° C. continued for a further 5 hours. The programmed water bath then returned to 6° C., and was maintained at that temperature overnight.

Samples were then stained with anti-mouse CD4-CyChrome (Pharmingen) and anti-mouse CD8 biotin (Immunotech). Samples were washed, and stained with streptavidin-ECD. Samples were washed and 100 μl of Fixative was added from the “Intraprep Permeabilization Reagent” kit (Immunotech) for 15 minutes at room temperature. After washing, 100 μl of permeabilization reagent from the Intraprep kit was added to each sample with anti-IFN-γ-PE+anti-IL-2-FITC. Samples were incubated at room temperature for 15 minutes, and washed. Samples were resuspended in 0.5 ml buffer, and analysed on the Flow Cytometer.

A total of 500,000 cells were collected per sample and subsequently CD4 and CD8 cells were gated to determine the populations of cells secreting IFNγ and/or IL-2 in response to stimulus.

The results show that all the polyproteins encoding Core, NS3, NS4B and NS5B in different orders are able to stimulate immune responses to NS3 (ie HCV 500, 510, 520, 530). The results are shown in FIG. 13. Responses to NS3 protein were similar between each of the HCV polyproteins (HCV 500, 510, 520 and 530), when monitored by IL2 (FIG. 13A) and IFNγ (FIG. 13B) ELISPOT.

The phenotype of the responding cells was analysed in more detail by ICS. A good CD4+ T cell response was elicited to an immunodominant NS3 CD4 specific peptide, which was similar between HCV 500, 510, 520, 530.

TABLE 1
Frequency of NS3 specific CD4 and CD8 T cells producing
IFNγ following immunisation with HCV polyproteins

| Construct | nil | NS3 protein | NS3 CD4 peptide | NS3 CD8 Peptide |
|---|---|---|---|---|
| NS3 single | 0.05 | 0.29 | 0.24 | 4.4 |
| HCV 500 | 0.09 | 0.27 | 0.38 | 5.54 |
| HCV 510 | 0.1 | 0.17 | 0.29 | 3.95 |
| HCV 520 | 0.1 | 0.14 | 0.28 | 3.32 |
| HCV 530 | 0.07 | 0.15 | 0.21 | 4.89 |
| HCV 501 | 0.1 | 0.05 | 0.08 | 0.16 |

IFNγ specific T cell responses were detected following of stimulation of splenocyt sin presence or absence of antigen for 6 hours, in presence of Brefeldin A for last 4 hours. IFNg was detected by gating on CD4 or CD8 T cells and staining with IFNγFITC.

A strong CD8 response to the immunodominant NS3 specific peptide was also generated following immunisation with HCV 500, 510, 520 and 530, reaching frequencies of between 2.5-6% of CD8+ cells.

Immunisation with HCV 500, 510, 520 and 530 also resulted in detection of CD4 and CD8 responses to both NS4B and NS5B antigens, although the CD8 responses were weaker to the polyproteins than following immunisation with the single antigen.

TABLE 2
Frequency of NS5B CD4 or CD8 specific T cells producing
IFNγ following immunisation with HCV polyproteins.

| | | | NS5B CD4 | |
|---|---|---|---|---|
| Plasmid | nil | NS5B protein | peptide | NS5B CD8 peptide |
| NS5B single | 0.05 | 0.1 | 0.26 | 1.67 |
| HCV 500 | 0.09 | 0.14 | 0.43 | 0.35 |
| HCV 510 | 0.11 | 0.1 | 0.29 | 0.11 |
| HCV 520 | 0.11 | 0.09 | 0.18 | 0.08 |
| HCV 530 | 0.07 | 0.06 | 0.7 | 0.12 |
| HCV 501 | 0.1 | 0.03 | 0.13 | 0.09 |

IFNγ specific T cell responses were detected following of stimulation of splenocytes in presence or absence of antigen for 6 hours, in presence of Brefeldin A for last 4 hours. IFNg was detected by gating on CD4 or CD8 T cells and staining with IFNγFITC.

TABLE 3
Frequency of NS4B CD4 or CD8 specific T cell producing
IFNγ following immunisation with HCV polyproteins.

| | | | NS4B CD4 | NS4B CD8 |
|---|---|---|---|---|
| Plasmid | nil | NS4B protein | peptide | peptide |
| NS4B | 0.05 | 0.17 | 0.18 | 2.04 |
| HCV500 | 0.09 | 0.09 | 0.1 | 0.6 |
| HCV510 | 0.05 | 0.09 | 0.09 | 0.34 |
| HCV520 | 0.06 | 0.08 | 0.05 | 0.33 |
| HCV530 | 0.1 | 0.17 | 0.1 | 0.37 |
| HCV501 | 0.04 | 0.09 | 0.06 | 0.13 |

IFNγ specific T cell responses were detected following of stimulation of splenocytes in presence or absence of antigen for 6 hours, in presence of Brefeldin A for last 4 hours. IFNg was detected by gating on CD4 or CD8 T cells and staining with IFNγFITC.

The peptides used have following sequence:

| | |
|---|---|
| Protein | Peptides |

| | |
|---|---|
| NS3 | (C57B1) |
| | CD4 PRFGKAIPIEAIKGG |
| | CD8 YRLGAVQNEVILTHP |
| NS5 | (C57BL/6). |
| | CD4 SMSYTWTGALITPCA |
| | CD8 AAALRAFTEAMTRYS |
| NS4B | (Balb/c) |
| | CD4 IQYLAGLSTLPGNPA |
| | CD8 FWAKHMWNFISGIWY |

### Recognition of Endogenously Processed Antigen

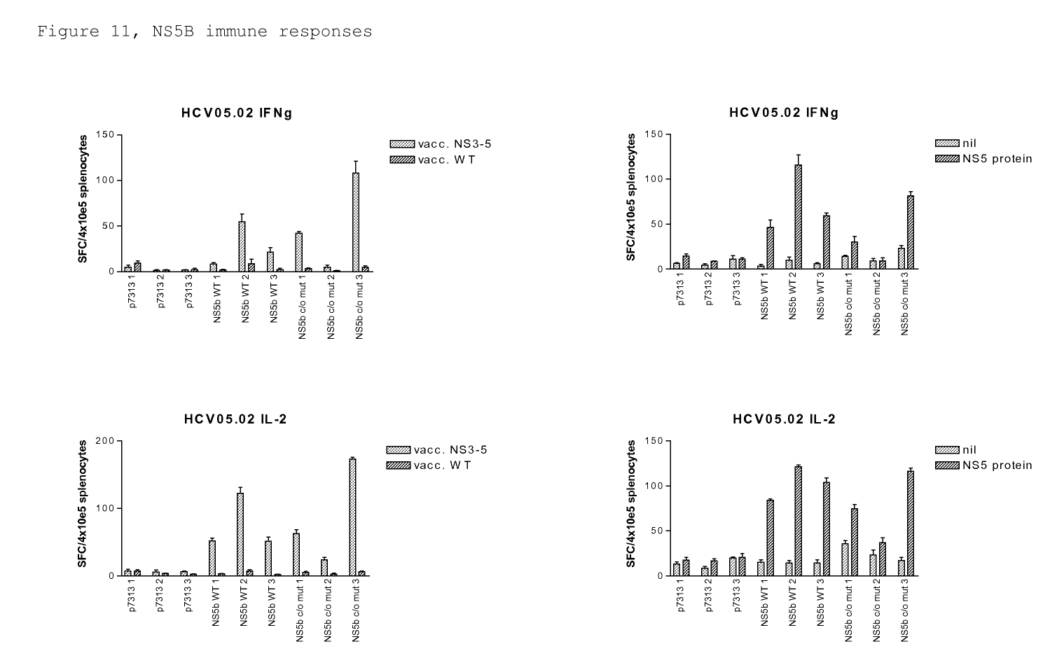

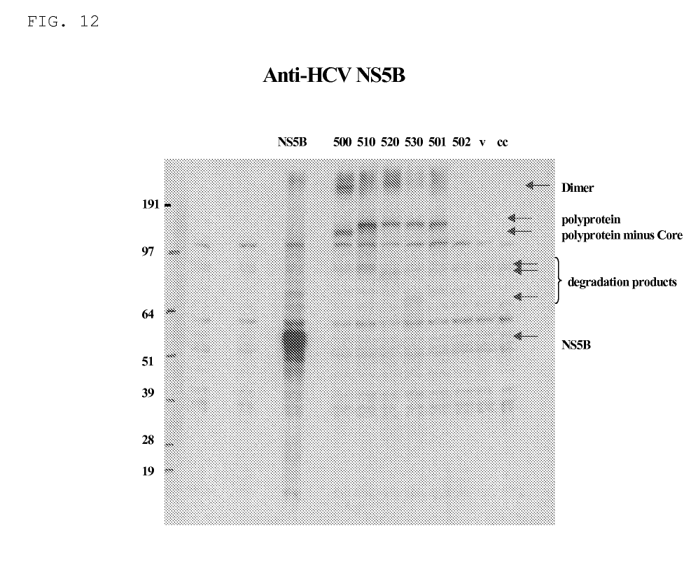

In order to determine if PMID immunisation with the HCV polyproteins induced a response that could recognise endogenously processed antigen, targets cells infected with Vaccinia recombinant virus expressing NS3-5 were used as stimulators in the ELISPOT assay. The results show that good IL2 and IFNγ ELISPOT responses were detected following immunisation with 500, 510, 520 and 530 (FIG. 14).

Immunisation with HCV Polyproteins Induces Functional CTL Activity.

C57BL mice were immunised with 0.01 μg DNA encoding NS3 alone, HCV 500, 510 and 520. Following a prime and a single boost, spleen cells from each group were re-stimulated in vitro with the NS3 CD8 peptide and IL2 for 5 days. CTL activity was measured against EL4 cells pulsed with the same peptide. Mice immunised with all constructs showed similar levels of killing in this assay.

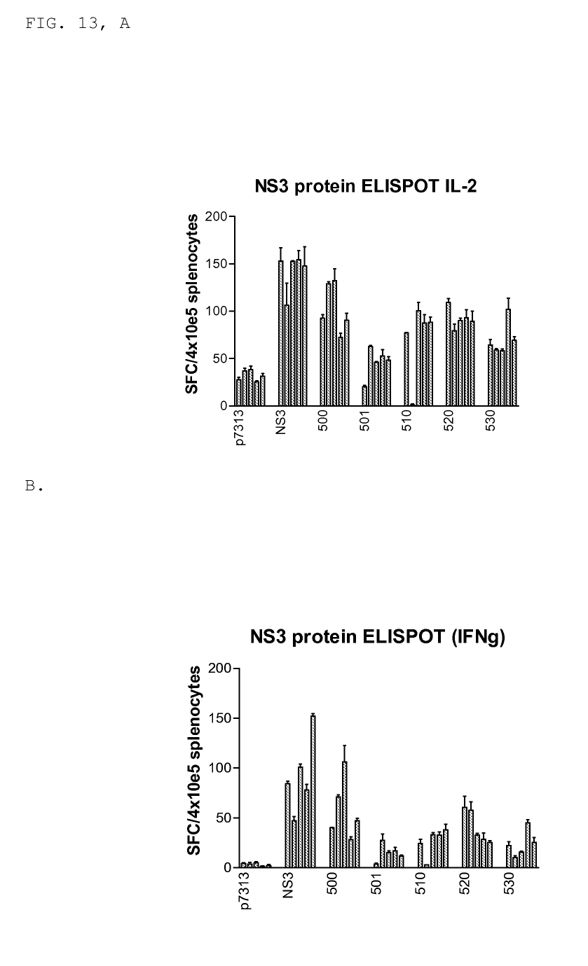

This shows that PMID immunisation with HCV polyproteins can induce functional CD8 responses. The results are shown in FIG. 15.

### EXAMPLE 6

### Delivery of HCV Antigens Via Dual Promoter Construct

Dual promoter constructs were generated using the following method. A fragment carrying expression cassette 1 (including Iowa-length CMV promoter, Exon 1, gene encoding protein/fusion protein of interest, plus rabbit globin poly-A signal) was excised from its host vector, namely p7313ie, by unique restriction endonuclease sites ClaI and XmnI. XmnI generates a blunt end at the 3-prime end of the excised fragment.

The recipient plasmid vector was p7313ie containing expression cassette 2. This was prepared by digest with unique restriction endonuclease Sse83871 followed by incubation with T4 DNA polymerase to remove the created 3-prime overhangs, resulting in blunt ends both 5-prime and 3-prime to the linear molecule. This was cut with unique restriction endonuclease ClaI, which removes a 259 bp fragment.

Expression cassette 1 was cloned into p7313ie/Expression cassette 2 via ClaI/blunt compatible ends, generating p7313ie/Expression cassette 1+Expression cassette 2, where cassette 1 is upstream of cassette 2.

p7313ie Plasmids comprising the following were generated

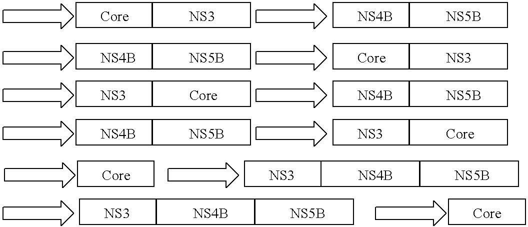

Footnote:
Arrow = Human Cytomegalovirus IE gene promoter (HCMV IE)
NS4B = truncated NS4B containing amino acids 49-260 - as outlined above.
Core = the Core protein containing amino acids 1-191.

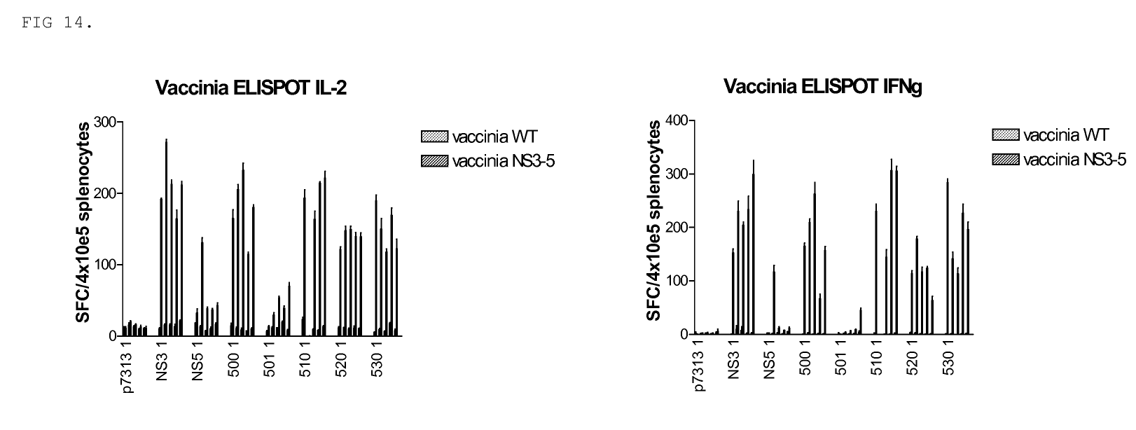

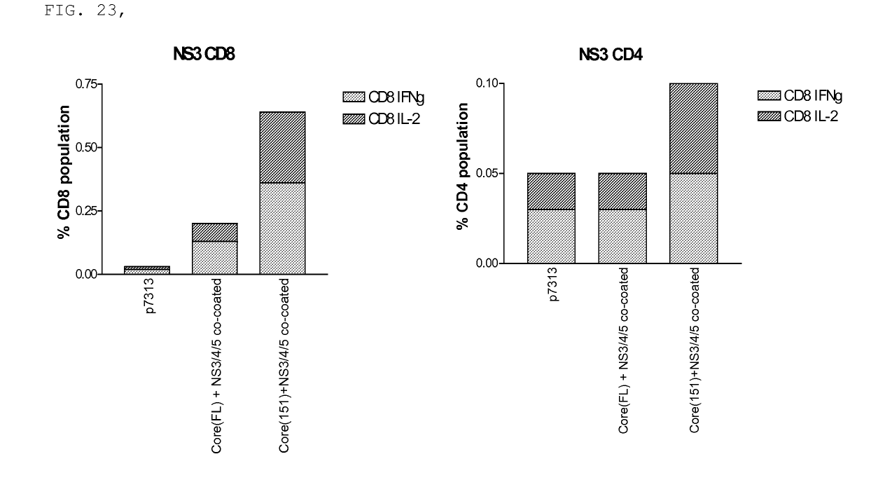

The construct panel shown above is complete and has been monitored for expression from transient transfection in 293T cells by Western blot. The results of the Western blot analysis are shown in FIG. 16: Lane key: NS3 were monitored 7 days post-boost, using intracellular cytokine staining to measure responses. The results shown in FIG. 23, show that both NS3 antigen specific CD4 and CD8 responses were approximately 2 fold high in the presence of Core 151 compared to Core 191.

Overall the in vivo studies comparing the response to NS3 in the presence of Core support the in vitro expression data that co-delivery of FL core and non-structural proteins can reduce expression of the non-structural antigens and this reduces the immunogenicity of the constructs. This effect can at least partially be overcome by co-coating with truncated core from which the C terminal 40 amino acids have been removed.

| | |
|---|---|
| 1. | p7313ie/Core |
| 2. | p7313ie/NS3 |
| 3. | p7313ie/NS5B |
| 4. | p7313ie/CoreNS3 |
| 5. | p7313ie/NS4B5B |
| 6. | p7313ie/NS3Core |
| 7. | p7313ie/NS34B5B |
| 8. | p7313ie/CoreNS3 + NS4B5B |
| 9. | p7313ie/NS4B5B + CoreNS3 |
| 10. | p7313ie/NS3Core + NS4B5B |
| 11. | p7313ie/NS4B5B + NS3Core |
| 12. | p7313ie/Core + NS34B5B |
| 13. | p7313ie/NS34B5B + Core |

Each pair of constructs carries two independent expression cassettes. It was not expected that the order in which the cassettes were inserted into the vector would have an effect upon the expression from either cassette. These results indicate, however, a significant disadvantage to the expression of NS4B5B or NS34B5B fusion proteins when their respective expression cassettes are positioned downstream of the Core, NS3Core, or CoreNS3 cassette.

Expression level is not as positive as for the single antigen constructs, however some reduction is to be expected due to the significant increase in size (175-228%), translating into a reduction in copy number of plasmid delivered to the cell by 50% for the same mass of DNA.

### In Vivo Immunogenicity Induced by Induced by Dual Promoter Constructs.

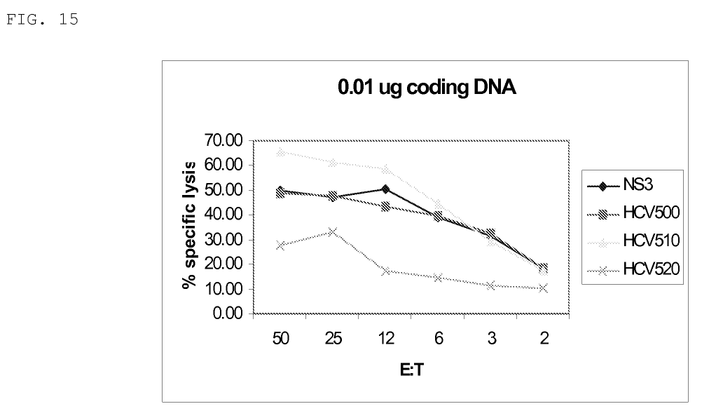

Three dual promoter constructs were selected for immunogenicity studies, which showed the greatest expression of all four antigens. These were p7313ie NS4B/NS5B+Core/NS3, p7313ieNS4B/NS5B+NS3Core and p7313ie NS3/NS4B/NS5B+Core. C57BL mice were immunised with 1 μg DNA by PMID and responses determined 7 days later to the dominant NS3 CD8 T cell epitope, using ELISPOT for IL2. The results (shown in FIG. 17) show that responses were observed to all three dual promoter constructs, after a single immunisation (Splenocytes stimulated with CD4 and Cd8 NS3 T cell specific peptides).

### EXAMPLE 7

### Deletion Mutation of Core

A number of genes encoding the ORF of Core, progressively deleted by a region spanning 20 amino acids per time from the 3′ end, were generated and fully sequenced.

| Core component | Nomenclature |
|---|---|
| 15-191 | Core Δ15 |
| 1-191 | Core 191 |
| 1-171 | Core 171 |
| 1-151 | Core 151 |
| 1-131 | Core 131 |
| 1-111 | Core 111 |
| 1-91 | Core 91 |
| 1-71 | Core 71 |
| 1-51 | Core 51 |

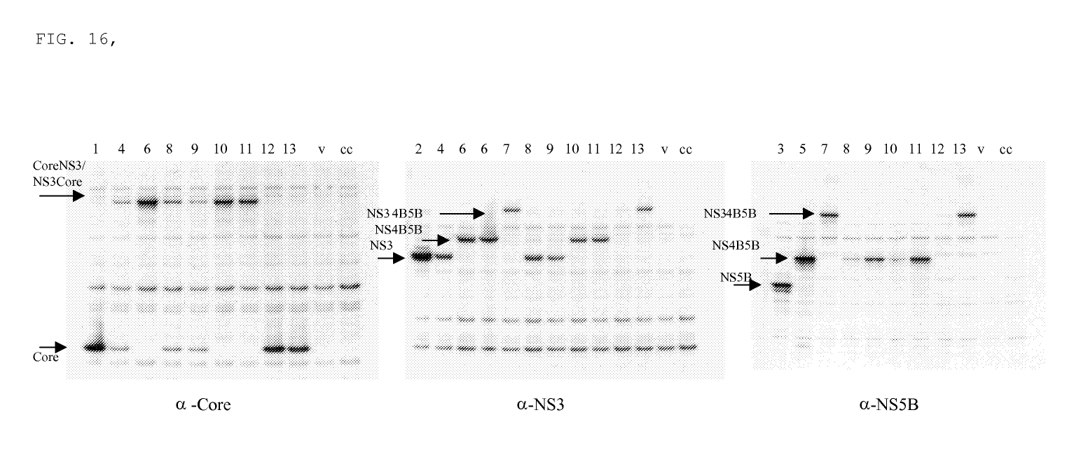

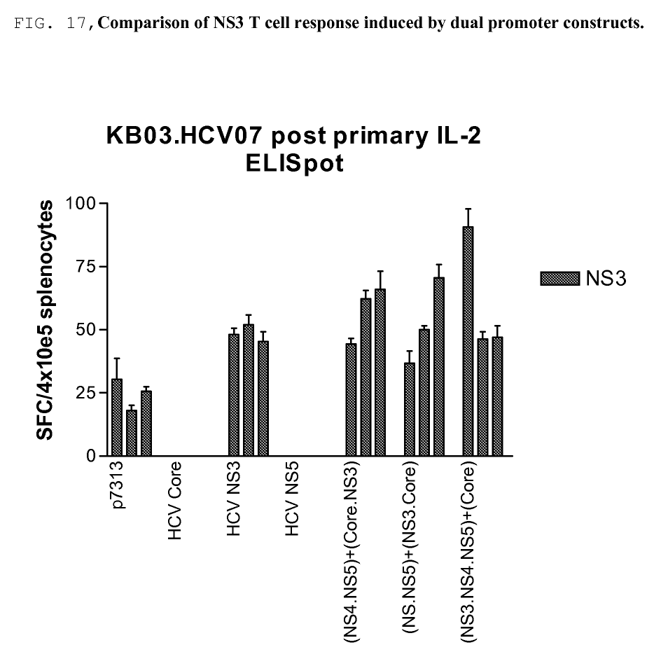

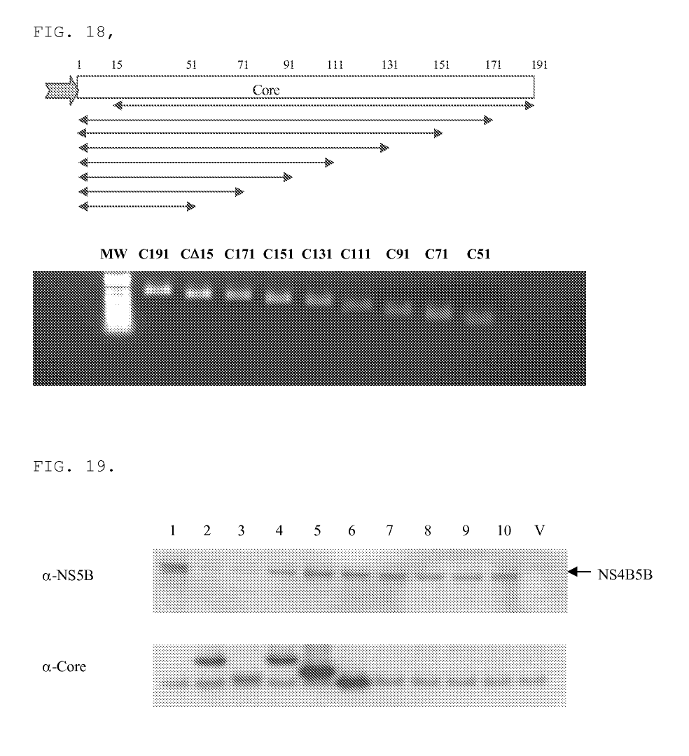

FIG. 18 depicts a DNA agarose gel showing the range of genes encoding fragments of Core. These constructs were tested for expression, combined with their effect upon the expression level of NS4B5B fusion (p7313ie/NS4B5B), by co-transfection in 293T cells. The results are shown in FIG. 19. The lanes being loaded as follows:

| | |
|---|---|
| | Loaded with (each |
| Lane | comprising 0.5 μg DNA) |

| | | |
|---|---|---|
| 1 | p7313ie/NS4B5B | p7313ie |
| 2 | p7313ie/NS4B5B | Core 191 |
| 3 | p7313ie/NS4B5B | Core Δ15 |
| 4 | p7313ie/NS4B5B | Core 171 |
| 5 | p7313ie/NS4B5B | Core 151 |
| 6 | p7313ie/NS4B5B | Core 131 |
| 7 | p7313ie/NS4B5B | Core 111 |
| 8 | p7313ie/NS4B5B | Core 91 |
| 9 | p7313ie/NS4B5B | Core 71 |
| 10 | p7313ie/NS4B5B | Core 51 |

The expression of Core191, Core Δ15, Core171, Core 151, and Core131 are clearly detected when the Western blot is probed with anti-Core, after anti-NS5B detection of the expression of NS4B5B. Further truncated forms of Core are not detected, possibly due to size capture restrictions of the gel system used.

The result demonstrates a significant reduction in expression level of NS4B5B in the presence of Core 191 and A 15, which recovers with Core 171, and again with Core 151, despite the strong expression of both Core species. This observation has been repeated twice with NS4B5B, and once with NS3 and NS5B.

### EXAMPLE 8

### Effect of Core and Core 151 Upon Expression of NS3, NS5B, an NS4B-NS5B Fusion and an NS3-NS4B-NS5B Triple Fusion

### Experiment 1

### Expression in Trans Format

An experiment was performed to monitor the effect of expression of Core 191 vs Core 151 upon the expression of the non-structural antigens, when Core is expressed in trans, or encoded on a separate plasmid. The experimental protocol was the same as that described in Example 7. Briefly, 0.5 μg each of two DNA plasmid vectors, outlined in the table below, were co-transfected into HEK 293T cells using Lipofectamine 2000 transfection reagent in a standard protocol (Invitrogen/Life Technologies). (Transfection and Western blot method as Example 4)

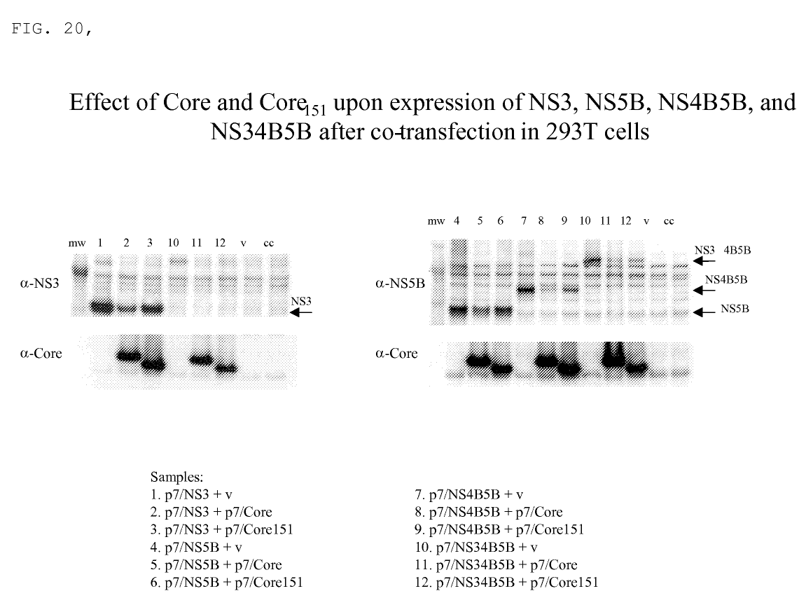

The results are shown in FIG. 20, where the lanes were loaded as described in the following table, and Western blot analysis was performed to detect the expression of non-structural proteins primarily, using anti-NS3 and anti-NS5B antisera, and that of Core by a secondary probe of the same blot with anti-Core.

| Lane | Non-structural element | Core element |
|---|---|---|
| 1 | NS3 | Empty vector |
| 2 | NS3 | Core 191 |
| 3 | NS3 | Core 151 |
| 4 | NS5B | Empty vector |
| 5 | NS5B | Core 191 |
| 6 | NS5B | Core 151 |
| 7 | NS4B-NS5B | Empty vector |
| 8 | NS4B-NS5B | Core 191 |
| 9 | NS4B-NS5B | Core 151 |
| 10 | NS3-NS4B-NS5B | Empty vector |
| 11 | NS3-NS4B-NS5B | Core 191 |
| 12 | NS3-NS4B-NS5B | Core 151 |

In all cases, the amount of non-structural protein or fusion (NS3, NS5B, NS4B-5B) when produced in trans with Core 151 has been demonstrated to be significantly increased in comparison with the level produced when expressed in trans with Core 191.

### Experiment 2

### Expression in Cis Format

An experiment was performed to monitor the effect of expression of Core 191 vs Core151 upon the expression of the non-structural antigens, when Core is expressed in cis, or encoded on the same plasmid in fusion with the non-structural elements. In each case, Core 151 was substituted for Core 191 in carboxy-terminal fusion with the non-structural region specified.

1 μg of DNA plasmid vector, outlined in the table below, was transfected into HEK 293T cells using Lipofectamine 2000 transfection reagent in a standard protocol (Invitrogen/Life Technologies). (Transfection and Western blot method as Example 4)

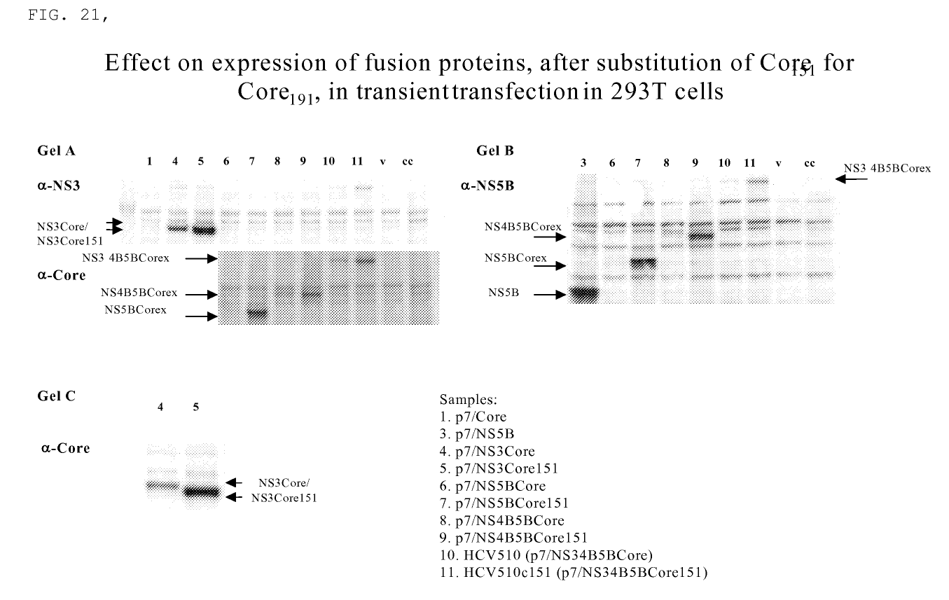

The results are shown in FIG. 21. Western blot analysis was performed to detect the expression of non-structural components primarily, using anti-NS3 and anti-NS5B antisera, and that of Core by a secondary probe of the same blot with anti-Core, in Gel A. The lanes were loaded as described in the following table:

| Lane | Non-structural element | Core element |
|---|---|---|
| 1 | — | Core 191 |
| 3 | NS5B | — |
| 4 | NS3 | Core 191 |
| 5 | NS3 | Core 151 |
| 6 | NS5B | Core 191 |
| 7 | NS5B | Core 151 |
| 8 | NS4B-NS5B | Core 191 |
| 9 | NS4B-NS5B | Core 151 |
| 10 | NS3-NS4B-NS5B (HCV 510) | Core 191 |
| 11 | NS3-NS4B-NS5B (HCV 510c) | Core 151 |

The results indicate that in a Cis format, where the antigens are in a polyprotein fusion, the truncation of Core increases the expression of the fusion protein.

### Comparison of Effect of Core191 and Core 151 on Immune Responses to NS3.

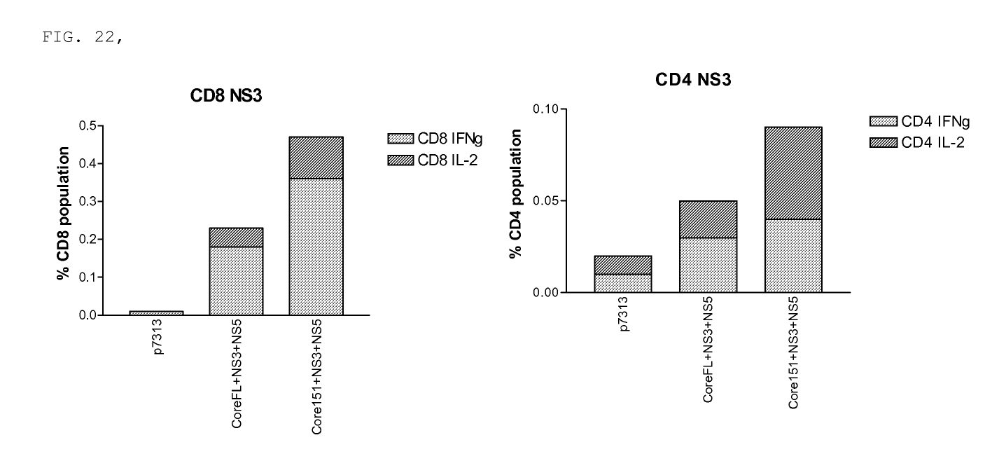

C57BL mice were immunised with 1.5 ug×2 shots total DNA by PMID. The groups immunised included empty vector p7313ie alone, co-coating of gold beads with p7313ieNS3, p7313ieNS5B and p7313ieCore 191 or p7313ieNS3, p7313ieNS5B and p7313ieCore151. Co-coating was used as this should deliver all plasmids to the same cell which should mimic the in vitro co-transfection studies described above. Immune responses to the dominant CD8 and CD4 T cell epitopes from NS3 were determined 14 days post primary immunisation using intracellular cytokine staining to measure IFNγ and IL2 antigen-specific responses. The results (shown in FIG. 22) show that both CD4 and CD8 NS3 responses were approximately 2 fold higher in the presence of Core151 compared to Core 191.

In another experiment C57BL mice were immunised with gold beads co-coated with plasmids expressing p7313ieNS3/NS4B/NS5B triple fusion together with either Core 191 or core 151. Animals were further boosted with the same constructs and responses to NS3 were monitored 7 days post-boost, using intracellular cytokine staining to measure responses. The results shown in FIG. 23, show that both NS3 antigen specific CD4 and CD8 responses were approximately 2 fold high in the presence of Core 151 compared to Core 191.

Overall the in vivo studies comparing the response to NS3 in the presence of Core support the in vitro expression data that co-delivery of FL core and non-structural proteins can reduce expression of the non-structural antigens and this reduces the immunogenicity of the constructs. This effect can at least partially be overcome by co-coating with truncated core from which the C terminal 40 amino acids have been removed.

1. An HCV vaccine comprising a polynucleotide that encodes the polypeptide sequences of the HCV proteins: core, NS3, NS4B and NS5B, for use in medicine.

2. An HCV vaccine as claimed in claim 1, wherein the polynucleotide encodes no other HCV protein.

3. An HCV vaccine as claimed in claim 1 or claim 2, wherein polynucleotide encodes a core protein which is truncated from the carboxy terminal end in a sufficient amount to reduce the inhibitory effect of Core upon the expression of other HCV proteins

4. An HCV vaccine as claimed in 3 wherein the truncated core protein has a deletion of at least the C-terminal 10 amino acids.

5. An HCV vaccine as claimed in claim 4 wherein the truncated core protein consists of the Core 1-151 sequence.

6. An HCV vaccine as claimed in claim 1, wherein the HCV proteins are present in the form of a fusion protein containing one or more of the HCV proteins.

7. An HCV vaccine as claimed in claim 6, wherein the fusion protein is a double fusion consisting of the polypeptide sequences of NS4B and NS5B.

8. An HCV vaccine as claimed in claim 6, wherein the fusion protein is a double fusion consisting of the polypeptide sequences of NS3 and Core

9. An HCV vaccine as claimed in claim 1, wherein the HCV proteins are encoded by the polynucleotide in more than one expression cassettes.

10. An HCV vaccine as claimed in claim 9, wherein the expression cassette encoding the Core protein is in a cis location downstream of the expression cassette which encodes at least on of the other HCV proteins.

11. An HCV vaccine as claimed in claim 10 wherein the expression cassette encoding the Core protein is downstream of an expression cassette which encodes the NS5B protein.

12. An HCV vaccine as claimed in claim 1, wherein at least one of the HCV proteins present are inactivated by mutation.

13. An HCV vaccine as claimed in claim 12, wherein the polynucleotide encodes a NS5B protein that comprises a mutation in motif A.

14. An HCV vaccine as claimed in claim 12, wherein the polynucleotide encodes a NS3 protein wherein the protease activity has been abrogated by mutation in any of the catalytic triad amino acids.

15. An HCV vaccine as claimed in claim 12, wherein the polynucleotide encodes a NS3 protein wherein the helicase activity has been abrogated by mutation in one or more of the helicase motifs I, II, III or IV.

16. An HCV vaccine as claimed in claim 12, wherein the polynucleotide encodes a NS4B protein comprising a truncation to remove the highly variable N-terminal region.

17. An HCV vaccine as claimed in any on of claims 1 to 16 wherein the polynucleotide vaccine encodes any one of the HCV combinations 1 to 19.

18. An HCV vaccine as claimed in claim 1, wherein the polynucleotide is a DNA sequence.

19. An HCV vaccine as claimed in claim 18 wherein the DNA sequence is in the form of a plasmid.

20. A vaccine as claimed in any one of claims 1 to 17 wherein the oligonucleotides are codon optimised for expression in mammalian cells.

21. A method of preventing or treating an HCV infection in a mammal comprising administering a vaccine as claimed in any one of claims 1 to 17 to a mammal.

22. A method of vaccination of an individual comprising taking a polynucleotide vaccine as claimed in any one of claims 1 to 17, coating the polynucleotide onto gold beads and delivering the gold beads into the skin.

23. Use of an HCV vaccine as claimed in any one of claims 1 to 17 in the manufacture of a medicament for the treatment of HCV.
